(19) United States
(12) Patent Application Publication
(10) Pub. No.: US 2025/0054631 A1
(43) Pub. Date: Feb. 13, 2025

(54) HEPATOCELLULAR CARCINOMA MOLECULAR SUBTYPE CLASSIFICATION AND SUBTYPE SPECIFIC TREATMENTS FOR HEPATOCELLULAR CARCINOMA

(71) Applicant: Genentech, Inc., South San Francisco, CA (US)

(72) Inventors: Yinghui GUAN, South San Francisco, CA (US); Shan LU, San Francisco, CA (US); Yulei WANG, South San Francisco, CA (US); Alexander ABBAS, South San Francisco, CA (US)

(21) Appl. No.: 18/929,486

(22) Filed: Oct. 28, 2024

Related U.S. Application Data
(63) Continuation of application No. PCT/US2023/020312, filed on Apr. 28, 2023.
(60) Provisional application No. 63/373,696, filed on Aug. 26, 2022.
(60) Provisional application No. 63/337,007, filed on Apr. 29, 2022.

(51) Int. Cl. G16H 50/20; G16H 20/17; G16H 30/40; G16H 50/70

(52) U.S. Cl. CPC G16H 50/20; G16H 20/17; G16H 30/40; G16H 50/70

(57) ABSTRACT

A method for hepatocellular carcinoma (HCC) subtype classification and treatment may include identifying, for a liver epithelial cell lineage, one or more features associated with the liver epithelial cell lineage. The one or more features may be designated as representative of a molecular subtype associated with hepatocellular carcinoma (HCC) such as, for example, a cholangio-like subtype, a hepatocyte-like subtype, or a progenitor-like subtype. A patient may be determined to exhibit the molecular subtype if these features are detected within the tumor sample of the patient. Moreover, treatment for the patient may be determined based on the molecular subtype exhibited by the patient. For example, treatment for the patient may include additional therapies, such as an GPC3/CD3 bi-specific antibody, to overcome subtype-specific resistance to combination immunotherapy associated with the progenitor-like subtype. Related systems and computer program products are also provided.

### CROSS-REFERENCE TO RELATED APPLICATIONS

This application is a continuation of International Application No. PCT/US2023/020312, filed on Apr. 28, 2023, which claims priority to and the benefit of U.S. Provisional Application No. 63/337,007, filed on Apr. 29, 2022, and U.S. Provisional Application No. 63/373,696, filed on Aug. 26, 2022, the entire contents of both of which are hereby incorporated by reference for all purposes.

### TECHNICAL FIELD

The subject matter described herein relates generally to digital pathology and more specifically to hepatocellular carcinoma (HCC) molecular subtype classification and subtype specific treatments for hepatocellular carcinoma.

### INTRODUCTION

Hepatocellular carcinoma (HCC) is a common and highly lethal malignancy. Combination therapy with atezolizumab (anti-PD-L1) and bevacizumab (anti-VEGF) has become the new standard care as a first-line therapy for hepatocellular carcinoma by demonstrating strong antitumor activity in clinical trials. However, nearly a third of patients still had progressive disease, highlighting a great need for understanding the tumor heterogeneity of hepatocellular carcinoma and mechanisms of response or resistance to guide novel treatment strategies.

### SUMMARY

Systems, methods, and articles of manufacture, including computer program products, are provided for hepatocellular carcinoma (HCC) subtype classification and treatment. In some example embodiments, there is provided a system that includes at least one processor and at least one memory. The at least one memory may include program code that provides operations when executed by the at least one processor. The operations may include: identifying, for a liver epithelial cell lineage, one or more features associated with the liver epithelial cell lineage; designating the one or more features as representative of a molecular subtype associated with hepatocellular carcinoma (HCC); receiving a tumor sample of a patient; and determining, based on the one or more features being detected within the tumor sample of the patient, that the patient exhibits the molecular subtype associated with hepatocellular carcinoma.

In another aspect, there is provided a method for hepatocellular carcinoma (HCC) subtype classification and treatment. The method may include: identifying, for a liver epithelial cell lineage, one or more features associated with the liver epithelial cell lineage; designating the one or more features as representative of a molecular subtype associated with hepatocellular carcinoma (HCC); receiving a tumor sample of a patient; and determining, based on the one or more features being detected within the tumor sample of the patient, that the patient exhibits the molecular subtype associated with hepatocellular carcinoma.

In another aspect, there is provided a computer program product including a non-transitory computer readable medium storing instructions. The instructions may cause operations may executed by at least one data processor. The operations may include: identifying, for a liver epithelial cell lineage, one or more features associated with the liver epithelial cell lineage; designating the one or more features as representative of a molecular subtype associated with hepatocellular carcinoma (HCC); receiving a tumor sample of a patient; and determining, based on the one or more features being detected within the tumor sample of the patient, that the patient exhibits the molecular subtype associated with hepatocellular carcinoma.

In some variations of the methods, systems, and non-transitory computer readable media, one or more of the following features can optionally be included in any feasible combination.

In some variations, the one or more features may include genetic features.

In some variations, a plurality of molecular subtypes associated with hepatocellular carcinoma (HCC) may be identified based at least on transcriptome data associated with a plurality of hepatocellular carcinoma (HCC) tissue samples.

In some variations, the plurality of subtypes may be identified by applying, to the transcriptome data, a cluster analysis to identify a quantity of subpopulations present within the transcriptome data.

In some variations, the cluster analysis may be applied to identify one or more subpopulations associated with a maximum cophenetic correlation value.

In some variations, the cluster analysis may include a non-negative matrix factorization (NMF).

In some variations, the cluster analysis may include one or more of a connectivity-based clustering, a centroid-based clustering, a distribution-based clustering, a density-based clustering, a subspace-based clustering, a group-based clustering, and a graph-based clustering.

In some variations, the plurality of subtypes may be identified and/or validated by applying, to the transcriptome data, a classifier.

In some variations, the classifier may include a random forest classifier.

In some variations, the one or more features may include at least one of a tumor-cell intrinsic feature and a tumor microenvironment feature.

In some variations, the one or more features may include an immunohistochemistry of cytochromes P450, an expression level of cytochromes P450, a Hippo signaling pathway, and/or an expression level of YES-associated protein (YAP).

In some variations, the one or more features may include a quantity of fibroblast activation protein in stroma, a vessel density, a density of cluster of differentiate 8 (CD8) in epitumor, a quantity of MHCI+ tumor cells, a density of cluster of differentiate 8 (CD8) in epitumor, a density of PDL1+, a density of activated T cells, and/or a density of exhausted T cells.

In some variations, the molecular subtype associated with hepatocellular carcinoma may include a cholangio-like subtype. The liver epithelial cell lineage may include cholangiocytes.

In some variations, the molecular subtype associated with hepatocellular carcinoma may include a hepatocyte-like subtype. The liver epithelial cell linage may include hepatocytes.

In some variations, the molecular subtype associated with hepatocellular carcinoma may include a progenitor-like subtype. The liver epithelial cell lineage may include bi-potent progenitors.

In some variations, a treatment for hepatocellular carcinoma (HCC) may be determined based at least on the molecular subtype of the patient.

In some variations, the treatment for hepatocellular carcinoma (HCC) may include a combination immunotherapy based at least on the patient having a cholangio-like subtype or a hepatocyte-like subtype.

In some variations, the treatment for hepatocellular carcinoma (HCC) may include an atezolizumab (anti-PD-L1) plus bevacizumab (anti-VEGF) combination therapy based at least on the patient having a cholangio-like subtype or a hepatocyte-like subtype.

In some variations, the treatment for hepatocellular carcinoma (HCC) mau include, based at least on the patient having a progenitor-like subtype, one or more additional therapies to overcome a subtype-specific resistance to combination immunotherapy associated with the progenitor-like subtype.

In some variations, the treatment for hepatocellular carcinoma (HCC) may include, based at least on the patient having a progenitor-like subtype, an GPC3/CD3 bi-specific antibody in addition to a combination immunotherapy.

In some variations, the one or more features may include a cancer epithelium tissue, a necrosis tissue, and/or a normal tissue present in an image of the tumor sample.

In some variations, the one or more features may include a growth pattern present in an image of the tumor sample.

In some variations, the one or more features may include one or more cancer epithelial cells, fibroblast cells, endothelial cells, and normal cells present in an image of the tumor sample.

In some variations, the one or more features may include one or more hepatocellular carcinoma (HCC) hepatocyte-like cancer epithelial cells, hepatocellular carcinoma (HCC) cancer epithelial cells with Mallory Hyaline or globules, and hepatocellular carcinoma (HCC) heptoblast-like cancer epithelial cells.

In some variations, a response and/or a pathological response to a treatment for the patient may be determined based at least on the molecular subtype of the patient.

Implementations of the current subject matter can include, but are not limited to, methods consistent with the descriptions provided herein as well as articles that comprise a tangibly embodied machine-readable medium operable to cause one or more machines (e.g., computers, etc.) to result in operations implementing one or more of the described features. Similarly, computer systems are also described that may include one or more processors and one or more memories coupled to the one or more processors. A memory, which can include a non-transitory computer-readable or machine-readable storage medium, may include, encode, store, or the like one or more programs that cause one or more processors to perform one or more of the operations described herein. Computer implemented methods consistent with one or more implementations of the current subject matter can be implemented by one or more data processors residing in a single computing system or multiple computing systems. Such multiple computing systems can be connected and can exchange data and/or commands or other instructions or the like via one or more connections, including, for example, to a connection over a network (e.g. the Internet, a wireless wide area network, a local area network, a wide area network, a wired network, or the like), via a direct connection between one or more of the multiple computing systems, etc.

The details of one or more variations of the subject matter described herein are set forth in the accompanying drawings and the description below. Other features and advantages of the subject matter described herein will be apparent from the description and drawings, and from the claims. While certain features of the currently disclosed subject matter are described for illustrative purposes in relation to hepatocellular carcinoma (HCC), it should be readily understood that such features are not intended to be limiting. The claims that follow this disclosure are intended to define the scope of the protected subject matter.

### DESCRIPTION OF DRAWINGS

The accompanying drawings, which are incorporated in and constitute a part of this specification, show certain aspects of the subject matter disclosed herein and, together with the description, help explain some of the principles associated with the disclosed implementations. In the drawings,

FIG. 1 depicts a system diagram illustrating an example of a digital pathology system, in accordance with some example embodiments;

FIG. 2 depicts a schematic diagram illustrating the liver epithelial cell lineage, tumor cell-intrinsic features, tumor microenvironment (TME) features, and immune landscape of different hepatocellular carcinoma (HCC) subtypes, in accordance with some example embodiments;

FIGS. 3A-3E depict the relationship between molecular subtypes and tumor-intrinsic and tumor microenvironmental heterogeneity in advanced hepatocellular carcinoma (HCC), in accordance with some example embodiments. Results shown in FIGS. 3A-3E are from 99 hepatocellular carcinoma samples in the training data set. FIG. 3A, experimental design of the study; FIG. 3B, cophenetic correlation of 1-5 clusters from NMF analysis with top 3,000 most variably expressed genes; FIG. 3C, unsupervised hierarchical clustering with top 3,000 most variably expressed genes. Box plots are showing expression of known liver epithelial cell lineage markers among NMF subtypes. Significant p-values from unpaired two-tailed t-test are shown between groups; FIG. 3D, hallmark gene set enrichment (GSEA) analysis showing unique pathway enrichment of each subtype against the other two; FIG. 3E, box plots are showing expression of curated signatures representing pathways signifying each subtype. Significant p-values from unpaired two-tailed t-test are shown between groups;

FIGS. 4A-4C depict cell lineage linked molecular subtype features being recapitulated in hepatocellular carcinoma and validated in situ, in accordance with some example embodiments. FIG. 4A pie charts comparing prevalence (% of total) of molecular subtypes in training data set, GO30140 arm A and IMbrave 150 biomarker population (BEP); FIG. 4B, expression profile of 288 core gene set ordered by subtypes classified by developed random forest classifiers and overlaid with etiology, PD-L1 IHC status and TP53 and CTNNB1 alterations in 90 GO30140 Arm A baseline samples; FIG. 4C, expression profile of 288 core gene set ordered by subtypes and overlaid with etiology, PD-L1 IHC status and TP53 and CTNNB1 alterations in 177 IMBrave 150 baseline samples;

FIGS. 5A-5C depict the relationship between molecular subtypes, Response Evaluation Criteria in Solid Tumours (RECIST) response, and benefit of atezolizumab plus bevacizumab over sorafenib, in accordance with some example embodiments. FIG. 5A, waterfall plot showing % maximum changes in sizes of longest dimension (SLD) from baseline target lesion ordered by subtype and magnitude of change; FIG. 5B, KM curve showing PFS of 3 subtypes in GO30140 Arm A; FIG. 5C, PFS (top row) and OS KM curves within subtypes stratified by treatments Atezo+Bev vs sorafenib. Hazard ratio and p-value are for the treatment arm term from Cox Proportional Hazards multivariate models that include terms for age, sex, MVI or EHS, AFP, and ECOG performance status;

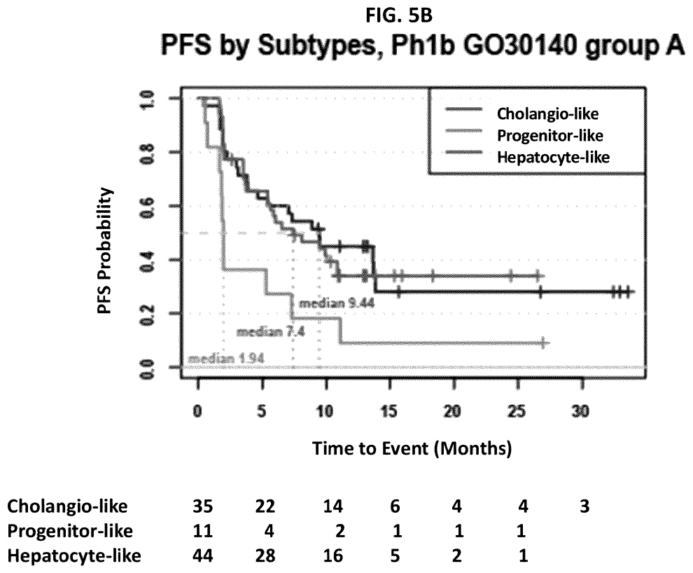

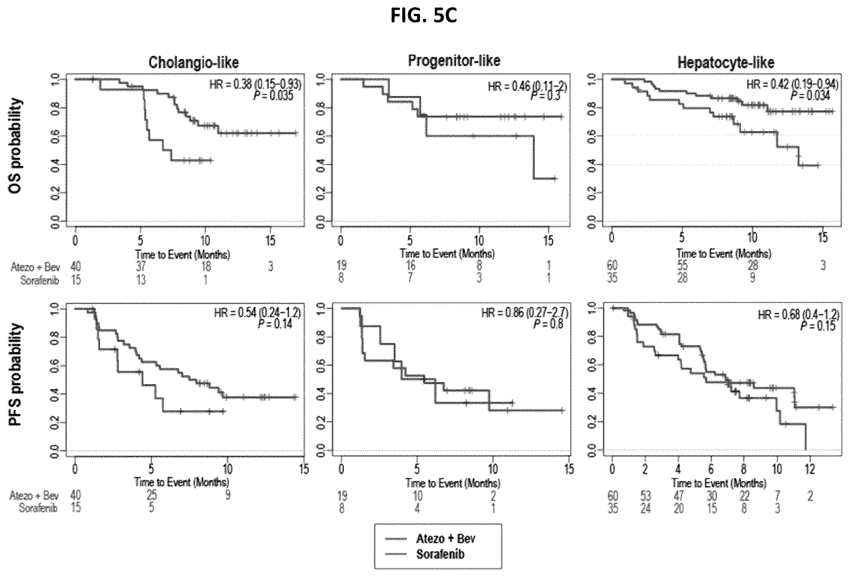

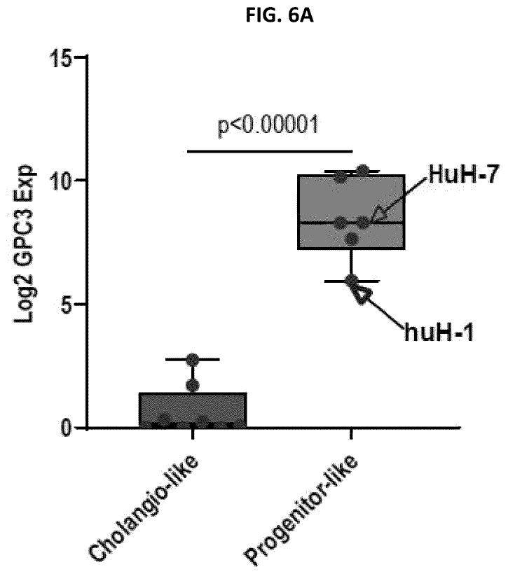

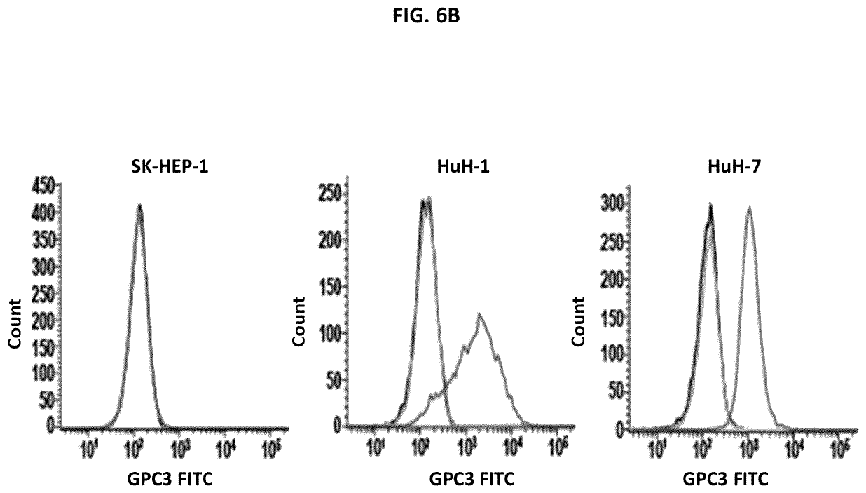

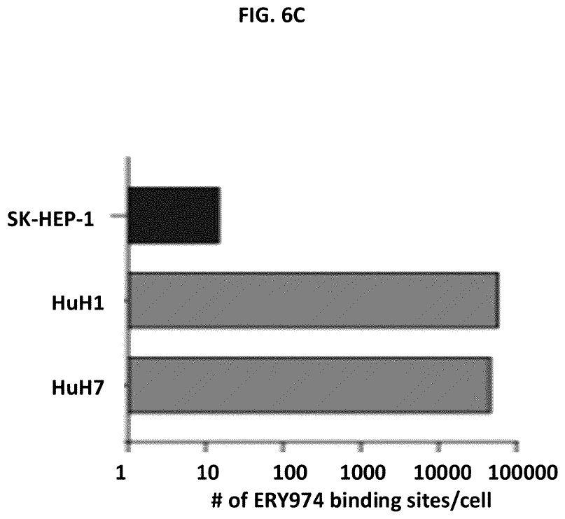

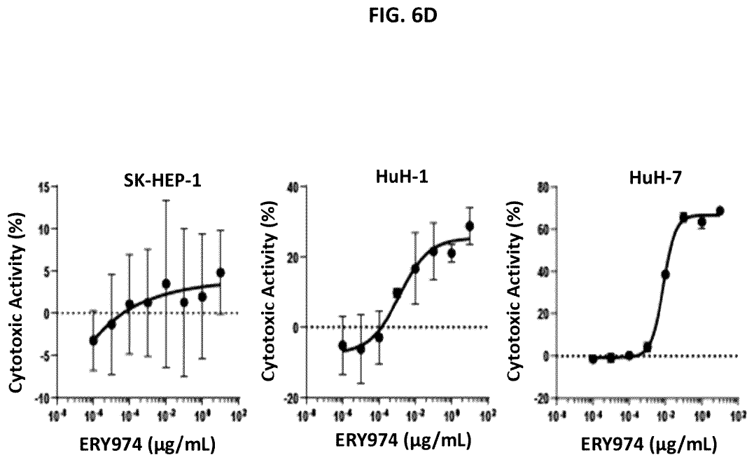

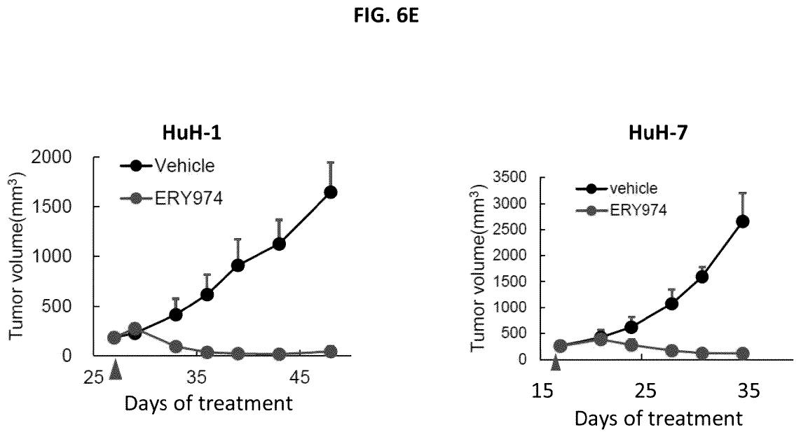

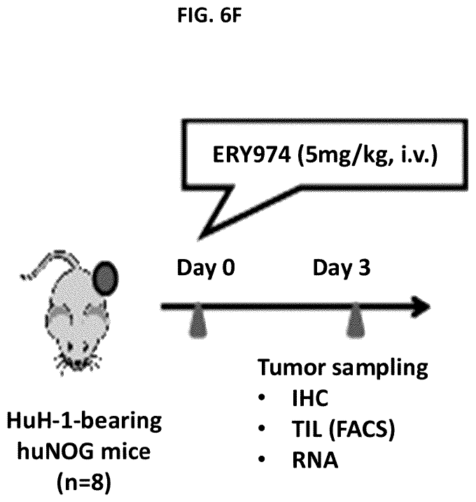

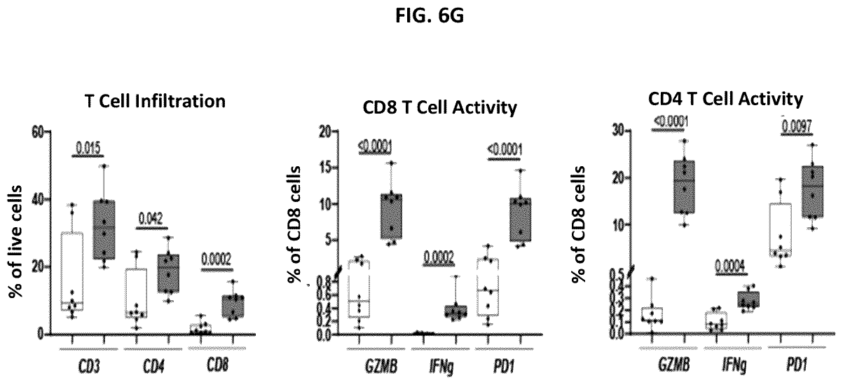

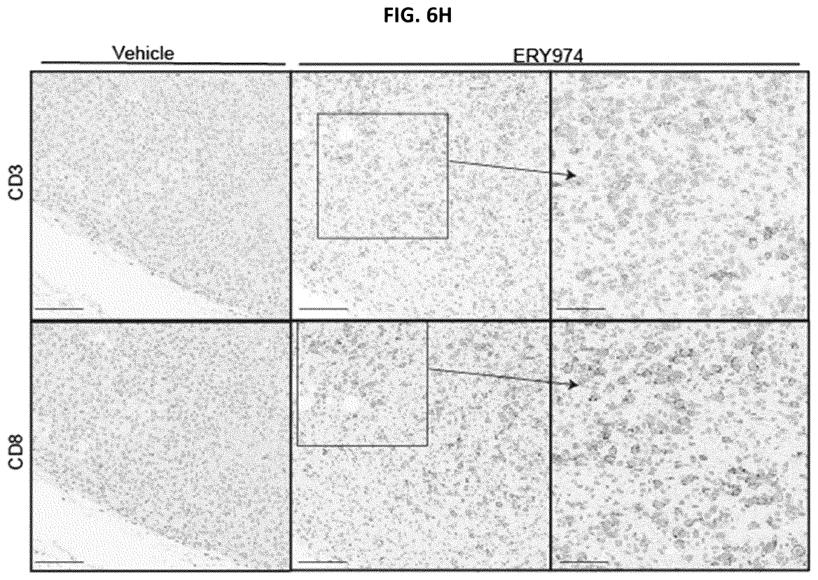

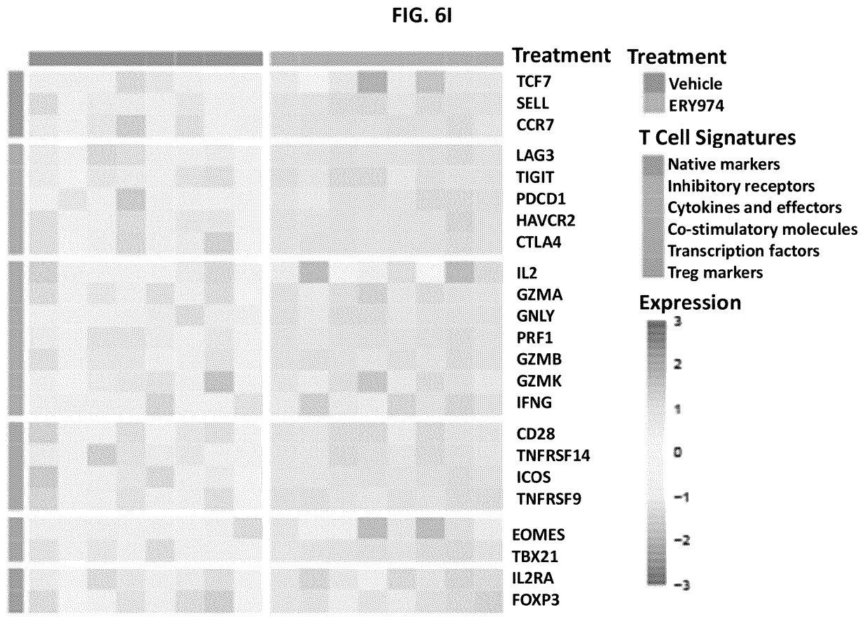

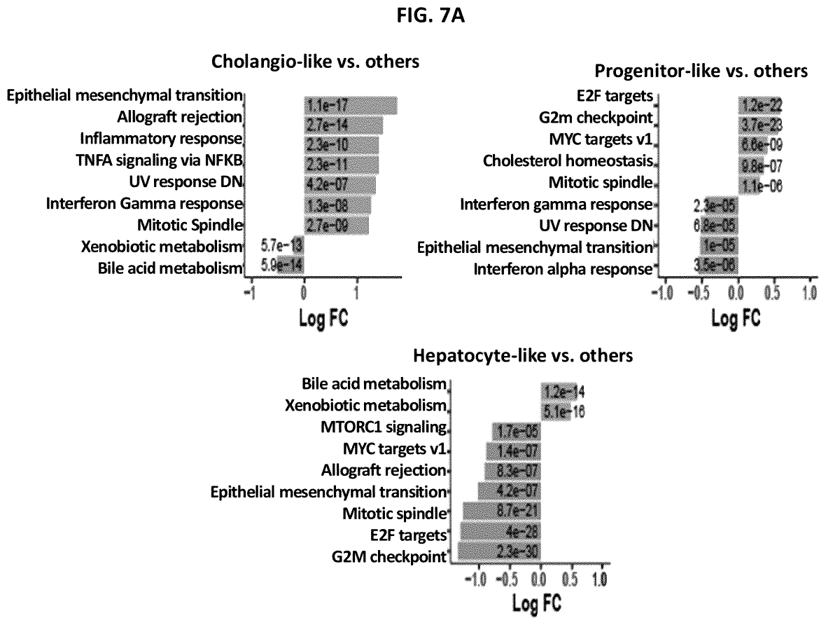

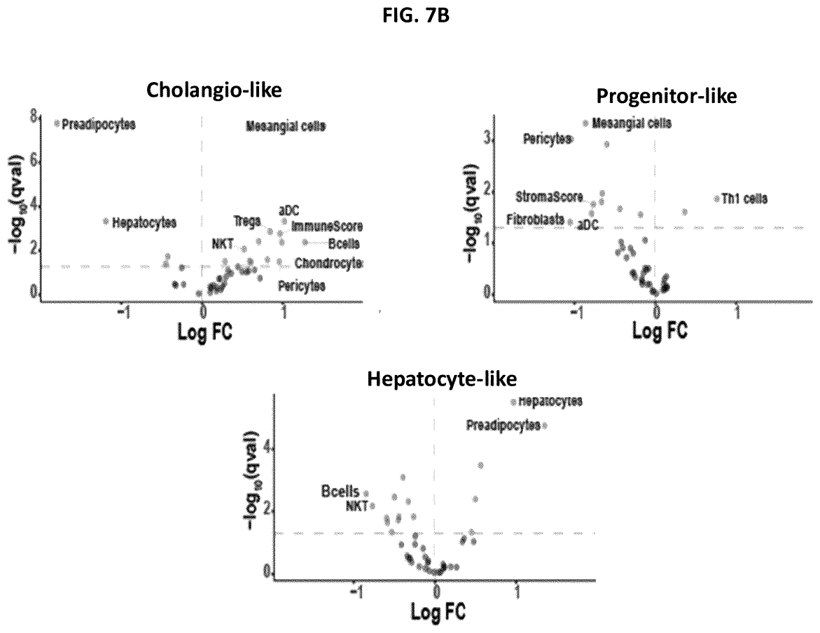

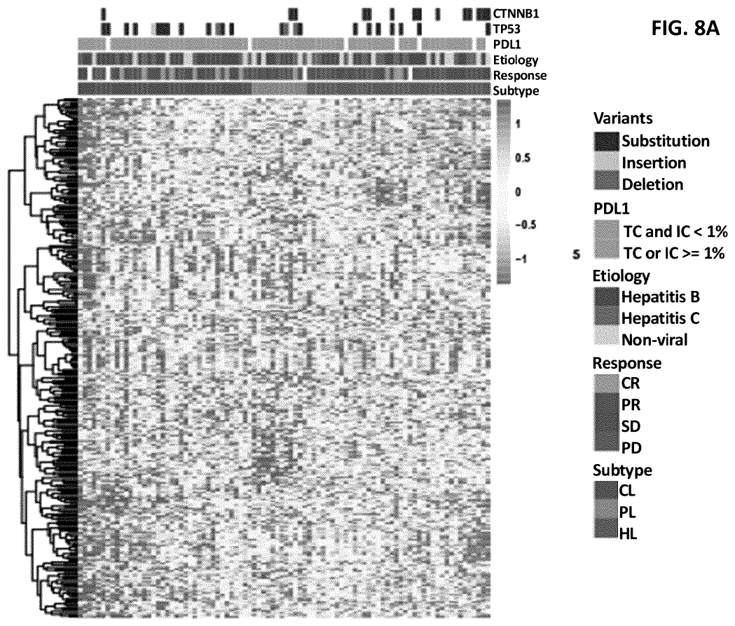

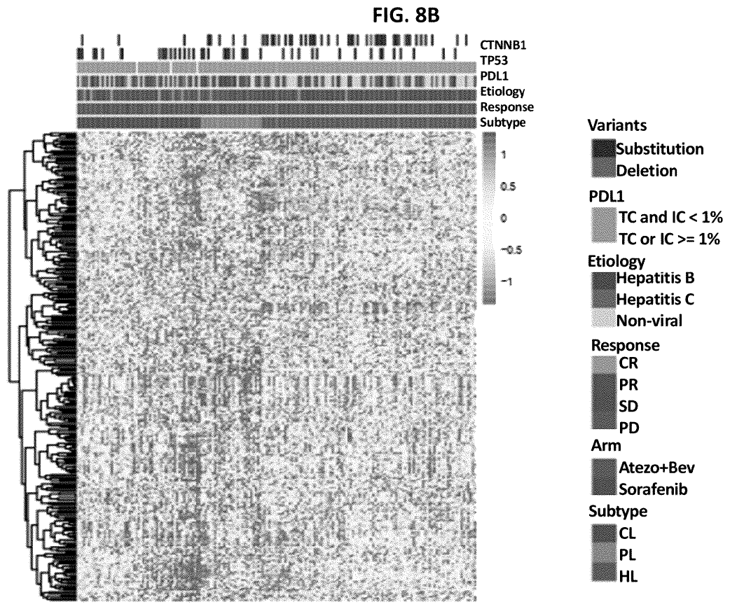

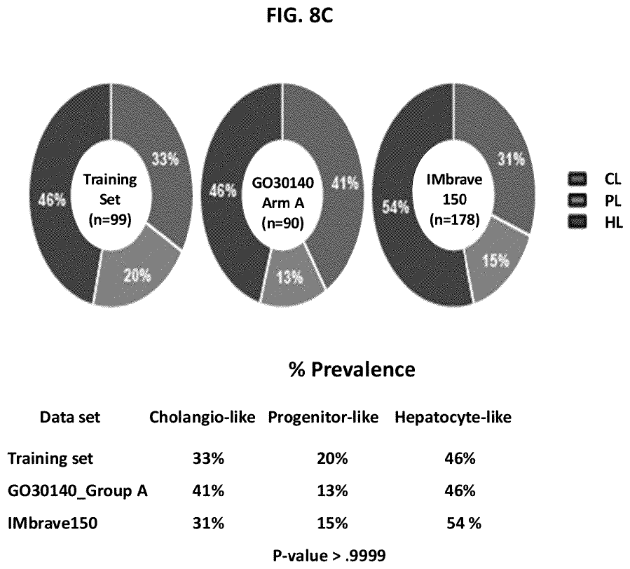

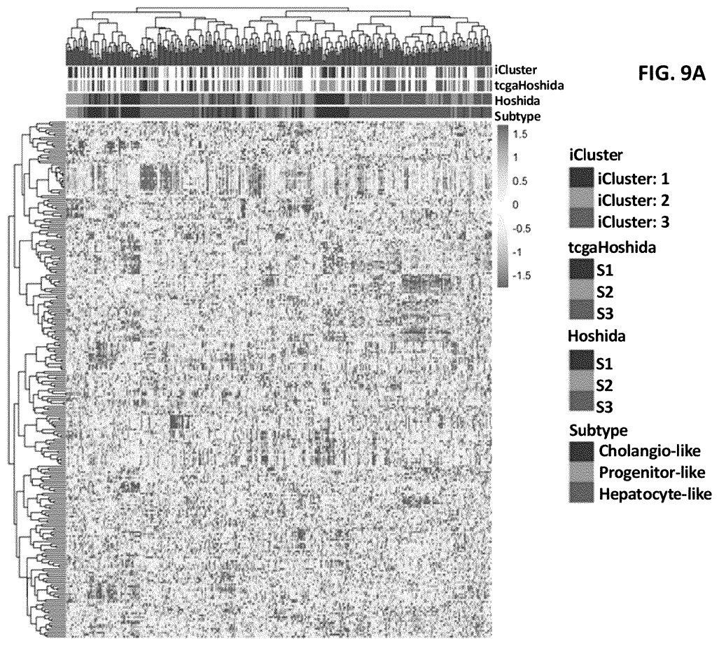

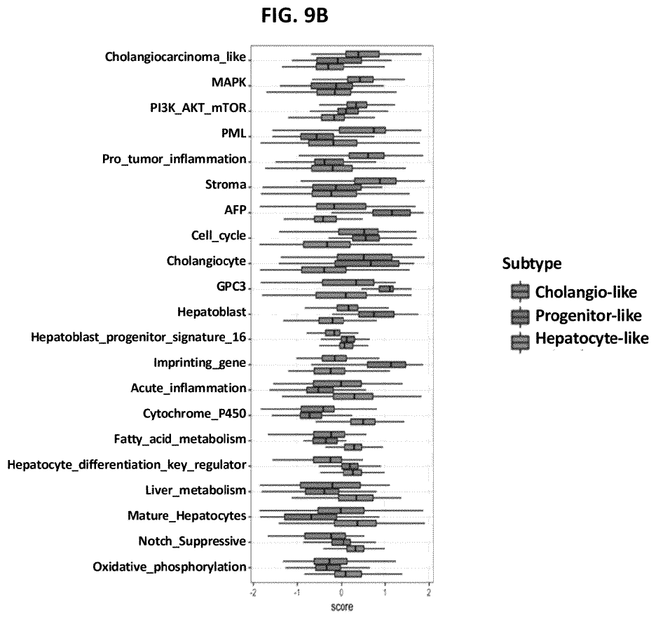

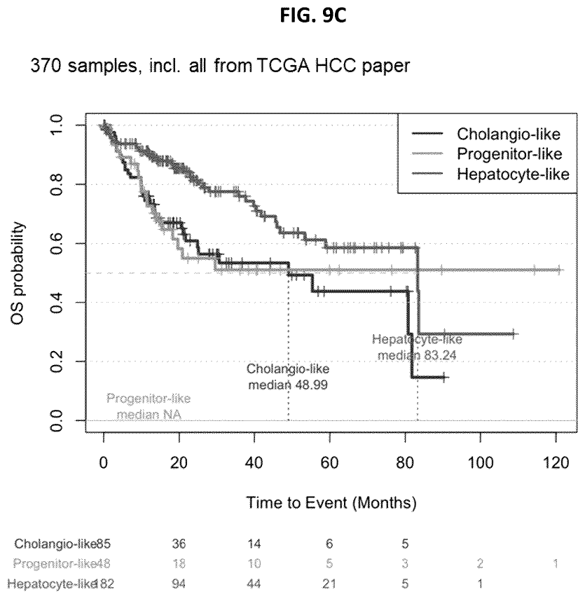

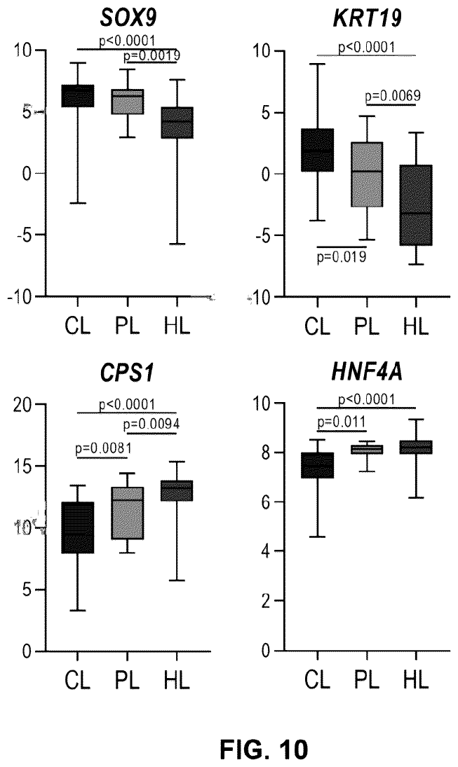

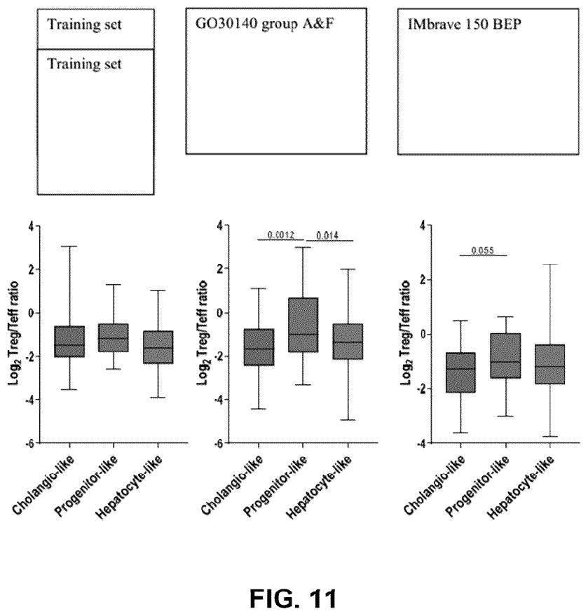

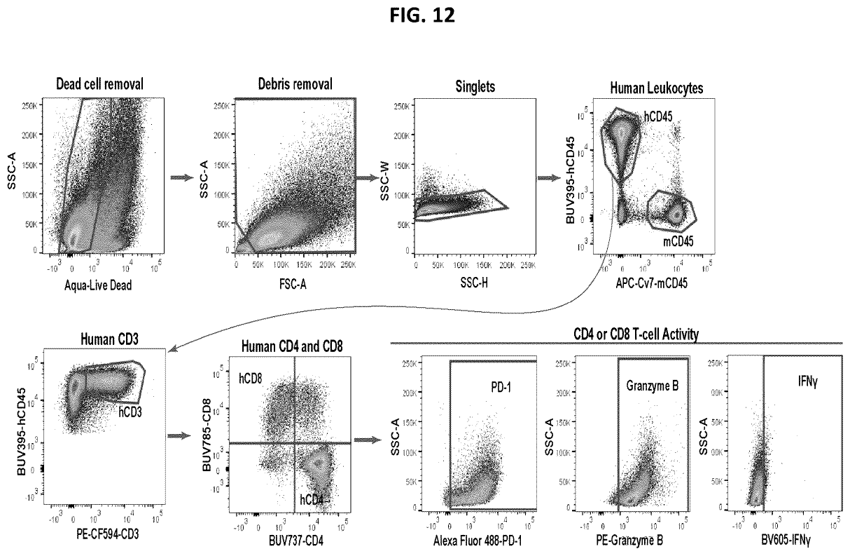

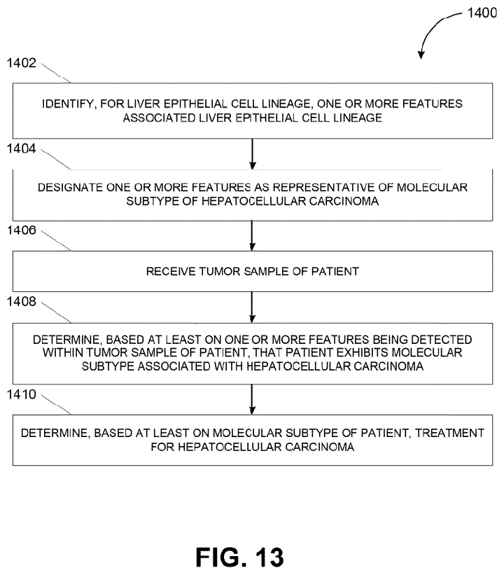

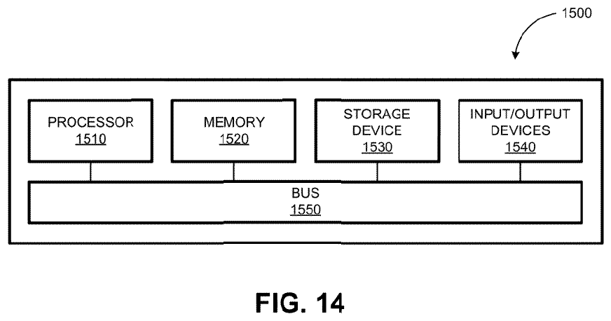

The data in FIGS. 5A-5C show that molecular subtypes of HCC may not only be characterized by distinct tumor intrinsic and tumor microenvironment features, they may also be clinically relevant and associated with different outcome from immunotherapies, e.g., cholangio-like subtype was associated with the most benefits from the combination therapy, while the progenitor-like subtype with high expression of GPC3 may appear to be less benefited. In some embodiments, biological insights into HCC heterogeneity and strategies of targeting subtype-specific vulnerabilities to overcome resistance to the combination immunotherapy are provided.

FIGS. 6A-6I depicts the anti-tumor activity of ERY974 in progenitor-like tumor being associated with increased T cell infiltration and cytolytic activity, in accordance with some example embodiments. FIG. 6A, GPC3 mRNA level in Cholangio (cholangio-like) cells and Progenitor (progenitor-like) cells. Statistical significance was determined by two-tailed unpaired t-test; FIG. 6B, Cell surface expression of GPC3 determined via flow cytometry with anti-GPC3 antibody. Black line indicates a shift with no antibody; green line indicates a shift with isotype control; red line indicates a shift with anti-GPC3 antibody; FIG. 6C, Antibody binding capacity (ABC) of GPC3 in SK-HEP1 (cholangio-like), huh-1 (progenitor-like), and huh-7 (progenitor-like). FIG. 6D, T cell dependent cytotoxicity assay (TDCC); FIG. 6E, antitumor efficacy of ERY974 in huh-1/huNOG (left) and huh-7/T cell injected (right)models (n=5). Dosage of ERY974 is 5 mg/kg in huh-1/huNOG, model and 1 mg/kg in huh-7/T cell injected model. The red arrow indicates the timing of ERY974 administration. TGI value is shown in each figure. Data is shown as the mean±standard deviation (SD) (n=5). p values are derived from two-tailed unpaired t-tests. n.s., no significance (Wilcoxon test); FIG. 6F, study design of PD analysis; FIG. 6G, tumor infiltrating lymphocyte analysis of huh-1 tumors on day 3 after ERY974 administration by flow cytometry (n=8 each group). Significant p values from unpaired two-tailed t-test between vehicle and ERY97 treated groups are shown; FIG. 6H, histopathological analysis of huh-1 tumor treated with ERY974 (on day 3). Representative HE staining (left), CD3 IHC (top row, middle and right), and CD8 IHC (bottom row, middle and right) are shown. The scale bars within images in left and middle columns are 100 μm and the ones in images in the right column are 50 μm. The rectangle insets within the middle column of images represent corresponding fields magnified in the right column of images; FIG. 6I, heatmap analysis for T cell marker genes and T cell activation marker genes of huh-1 tumor RNA treated with ERY974 on day 3 (n=8);

FIGS. 7A-7B depict Gene Set Enrichment Analysis (GSEA) and xCell analysis of hepatocellular carcinoma molecular subtypes, in accordance with some example embodiments;

FIGS. 8A-8C depict hepatocellular carcinoma molecular subtypes being recapitulated in the GO30140 Arm A study and the IMBrave 150 study, in accordance with some example embodiments;

FIGS. 9A-9C depict the molecular subtypes and pathway enrichment present in The Cancer Genome Atlas (TCGA) hepatocellular carcinoma (HCC) dataset, in accordance with some example embodiments;

FIG. 10 depicts the expression of known hepatocellular carcinoma cell lineage marker genes in the training set, in accordance with some example embodiments;

FIG. 11 depicts a higher regulatory T-cell (T_{reg}) to effector T-cell (T_{eff}) ratio in the progenitor-like subtype, in accordance with some example embodiments;

FIG. 12 depicts a fluorescence-activated cell sorting (FACS) gating strategy for characterizing T-cell infiltration and activity in xenografted tumors, in accordance with some example embodiments;

FIG. 13 depicts a flowchart illustrating an example of a process for hepatocellular carcinoma (HCC) subtype classification and treatment, in accordance with some example embodiments;

FIG. 14 depicts a block diagram illustrating an example of a computing system, in accordance with some example embodiments.

When practical, similar reference numbers denote similar structures, features, or elements.

### DETAILED DESCRIPTION

Hepatocellular carcinoma (HCC) is a highly heterogeneous disease with complex etiological factors as well as diverse molecular and cellular dysfunctions. Several molecular classification of hepatocellular carcinoma have been determined based on gene expression signatures, genetic/epigenetic landscape, and metabolic networks. Interestingly, the heterogeneity of these subtypes are not only characterized by diverse molecular features including oncogenic pathways and immune cell infiltration patterns but also morphology and cell differentiation stages. Nevertheless, the clinical relevance of these hepatocellular carcinoma subtypes, especially in the context of immunotherapies, has not been well characterized. In fact, treatment options for hepatocellular carcinoma (HCC) remains a disease with poor prognosis and limited treatment options. Although combination immunotherapies, such as atezolizumab (anti-PD-L1) plus bevacizumab (anti-VEGF) combination therapy, have recently become the new standard of care in patients with unresectable hepatocellular carcinoma, not all patients derive benefit from these treatments. As such, gaining biological insights into hepatocellular carcinoma heterogeneity remains crucial for understanding the mechanisms of response and resistance to the combination immunotherapy and identifying effective new therapeutic targets.

In some example embodiments, a pathological response to a treatment (e.g., major pathological response (MPR) and/or the like), a response to a treatment, and/or one or more suitable treatments for hepatocellular carcinoma (HCC) may be determined based on the subtype of hepatocellular carcinoma present in a patient. In particular, three separate hepatocellular carcinoma molecular subtypes were identified through transcriptomic, genomic, and in situ analyses in three independent hepatocellular carcinoma cohorts including GO30140 Phase 1b and IMbrave150 phase 3 trials. The three subtypes, cholangio-like, progenitor-like, and hepatocyte-like, are identified and validated based on their linkage to different liver epithelial cell lineages. Moreover, each subtype is associated with distinct tumor cell-intrinsic features, tumor microenvironment (TME) features, and immune landscape. For example, the progenitor-like subtype is associated with bi-potent progenitors, the cholangio-like subtype is associated with cholangiocytes, and the hepatocyte-like subtype is associated with hepatocytes. Each subtype may exhibit different tumor cell-intrinsic features such as immunohistochemistry of cytochromes P450, expression level of cytochromes P450, Hippo signaling pathway, expression level of YES-associated protein (YAP), and/or the like. Furthermore, each subtype may exhibit different tumor microenvironment (TME) features including, for example, quantity of fibroblast activation protein in stroma, vessel density, density of cluster of differentiate 8 (CD8) in epitumor, quantity of MHCI+ tumor cells, density of cluster of differentiate 8 (CD8) in epitumor, density of PDL1+, density of activated T cells, density of exhausted T cells, and/or the like.

In some example embodiments, the aforementioned biological insights into hepatocellular carcinoma heterogeneity may be leveraged towards formulating subtype-specific treatment strategies including those that target subtype-specific vulnerabilities to overcome resistance to combination immunotherapy such as atezolizumab (anti-PD-L1) plus bevacizumab (anti-VEGF) combination therapy. That is, the molecular subtype exhibited by a hepatocellular carcinoma (HCC) patient may be indicative of the patient's pathological response (e.g., major pathological response (MPR) and/or the like) or response to one or more treatments for hepatocellular carcinoma. For example, patients exhibiting the cholangio-like subtype derive the most benefits from the combination immunotherapy whereas patients exhibiting the progenitor-like subtype benefit less. Accordingly, in some cases, treatment for a hepatocellular carcinoma (HCC) patient exhibiting the progenitor-like subtype may include a GPC3/CD3 bi-specific antibody in addition to combination immunotherapy in order to overcome the subtype-specific resistance to the combination immunotherapy associated with the progenitor-like subtype.

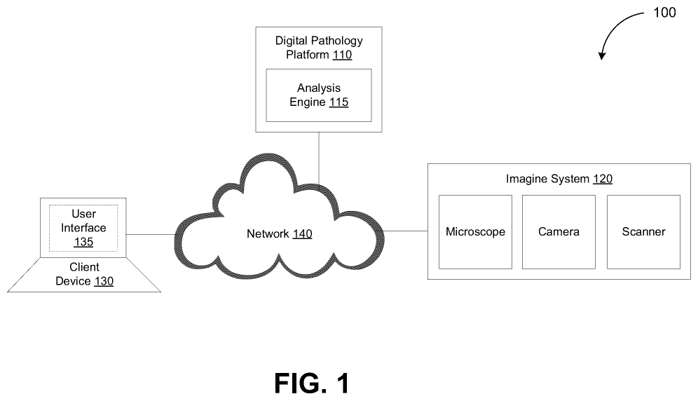

FIG. 1 depicts a system diagram illustrating an example of a digital pathology system 100, in accordance with some example embodiments. Referring to FIG. 1, the digital pathology system 100 may include a digital pathology platform 110, an imaging system 120, and a client device 130. As shown in FIG. 1, the digital pathology platform 110, the imaging system 120, and the client device 130 may be communicatively coupled via a network 140. The network 140 may be a wired network and/or a wireless network including, for example, a local area network (LAN), a virtual local area network (VLAN), a wide area network (WAN), a public land mobile network (PLMN), the Internet, and/or the like. The imaging system 120 may include one or more imaging devices including, for example, a microscope, a digital camera, a whole slide scanner, a robotic microscope, and/or the like. The client device 130 may be a processor-based device including, for example, a workstation, a desktop computer, a laptop computer, a smartphone, a tablet computer, a wearable apparatus, and/or the like.

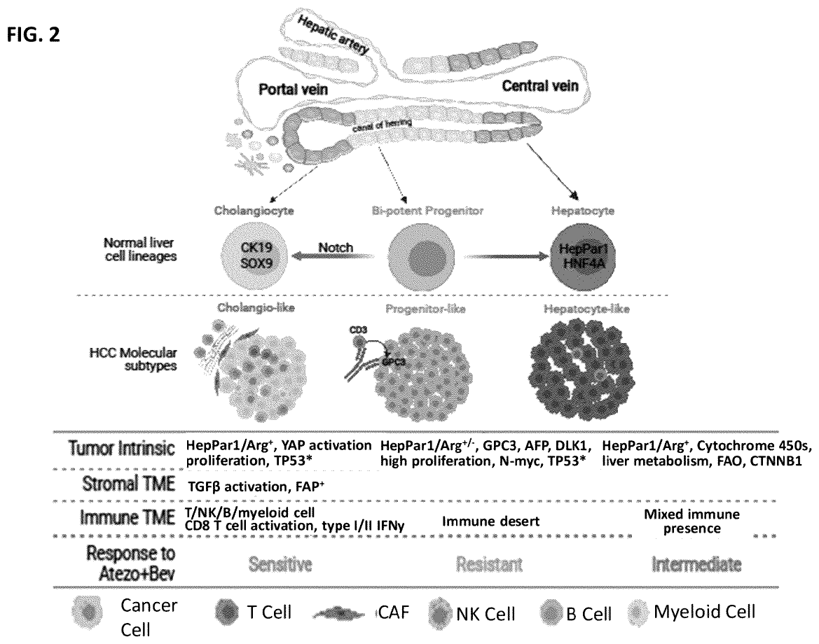

Referring again to FIG. 1, the digital pathology platform 110 may include an analysis engine 115 configured to determine, based on a hepatocellular carcinoma (HCC) tumor sample of a patient, a hepatocellular carcinoma molecular subtype exhibited by the patient. Moreover, the analysis engine 115 may determine, based at least on the hepatocellular carcinoma molecular subtype exhibited by the patient, a hepatocellular carcinoma (HCC) treatment for the patient. As FIG. 2 illustrates, hepatocellular carcinoma is a heterogeneous disease in which different molecular subtypes are strongly linked to liver epithelial cell lineage as well as unique tumor cell-intrinsic features, tumor microenvironment (TME) features, and immune landscape. Accordingly, the analysis engine 115 may determine, based at least on the hepatocellular carcinoma tumor sample of the patient, that the patients exhibits a progenitor-like subtype associated with bi-potent progenitors, a cholangio-like subtype is associated with cholangiocytes, or a hepatocyte-like subtype is associated with hepatocytes. In the event the patient is determined to exhibit the cholangio-like subtype or the hepatocyte-like subtype, the analysis engine 115 may determine a treatment that includes combination immunotherapy (e.g., an atezolizumab (anti-PD-L1) plus bevacizumab (anti-VEGF) combination therapy and/or the like). Alternatively, if the analysis engine 115 determines that the patient exhibits the progenitor-like subtype, the analysis engine 115 may determine a treatment that includes an GPC3/CD3 bi-specific antibody in addition to a combination immunotherapy in order to overcome a subtype-specific resistance to the combination immunotherapy.

In some example embodiments, different hepatocellular carcinoma (HCC) subtypes, such as the progenitor-like, cholangio-like, and hepatocyte-like molecular subtypes, may be identified based on transcriptome data. For example, the analysis engine 115 may identify the different hepatocellular carcinoma (HCC) subtypes by applying, to transcriptome data associated various hepatocellular carcinoma tumor samples, a cluster analysis such as non-negative matrix factorization (NMF), connectivity-based clustering, centroid-based clustering, distribution-based clustering, density-based clustering, a subspace-based clustering, group-based clustering, graph-based clustering, and/or the like. The cluster analysis may be applied to generate a quantity of clusters associated with a maximum cophenetic correlation value. In some cases, in addition to or instead of the cluster analysis, the analysis engine 115 may identify the different hepatocellular carcinoma (HCC) subtypes by applying, to transcriptome data associated various hepatocellular carcinoma tumor samples, a classifier (e.g., an ensemble learning model such as a random forest classifier and/or the like). For example, in some example embodiments, while the analysis engine 115 may apply a first technique (e.g., a cluster analysis) to identify the different hepatocellular carcinoma (HCC) subtypes, the results associated with the first technique may be validated by the analysis engine 115 applying a second technique (e.g., a classifier).

In some example embodiments, the different hepatocellular carcinoma (HCC) molecular subtypes identified based on transcriptome data may also be identified based on histological features present in images of hepatocellular carcinoma (HCC) samples. In some cases, the images of the hepatocellular carcinoma (HCC) samples may be whole slide images (WSI). In some cases, the images of the hepatocellular carcinoma (HCC) samples may have been treated, for example, by stains such as an hematoxylin and eosin (H&E) stain and/or the like. Accordingly, the analysis engine 115 may identify, within an image (e.g., a whole slide image (WSI) and/or the like) of a hepatocellular carcinoma (HCC) sample, a variety of features including, for example, different types of tissues, cells, growth patterns, and/or the like.

In some example embodiments, the analysis engine 115 may identify, within an image of a hepatocellular carcinoma (HCC) sample, one or more regions corresponding to different types of tissue. For example, the analysis engine 115 may differentiate and annotate regions corresponding to cancer epithelium tissue, necrosis tissue, normal tissue, and/or the like. Alternatively and/or additionally, the analysis engine 115 may differentiate and annotate various regions such as, for example, bile duct, benign lobules, and/or the like. In some cases, the analysis engine 115 may further identify a growth pattern for the hepatocellular carcinoma (HCC) present within the image of the hepatocellular carcinoma (HCC) sample including, for example, macrotrabecular, trabecular or sinusoidal, solid, pseudoacinar, and/or the like.

In some example embodiments, the analysis engine 115 may identify, within an image of a hepatocellular carcinoma (HCC) sample, one or more regions corresponding to different types of cells. For example, the analysis engine 115 may differentiate and annotate regions corresponding to cancer epithelial cells, fibroblast cells, endothelial cells, and normal cells (e.g., non-cancerous cells). Alternatively and/or additionally, the analysis engine 115 may differentiate and annotate regions corresponding to lymphocytes, plasma cells, and macrophages. In some cases, the analysis engine 115 may differentiate and annotate regions corresponding to cancer epithelial cells, fibroblast cells, endothelial cells, lymphocytes, plasma cells, macrophages, and normal cells (e.g., non-cancerous cells). Furthermore, in some cases, the analysis engine 115 may differentiate and annotate different types of cancer epithelial cells including, for example, hepatocellular carcinoma (HCC) hepatocyte-like, hepatocellular carcinoma (HCC) with Mallory Hyaline or globules, hepatocellular carcinoma (HCC) heptoblast-like (e.g., smaller cells exhibiting a high nucleus to cytoplasm ratio), and/or the like.

In some example embodiments, to identify a correlation between the features present within the image of the hepatocellular carcinoma (HCC) sample and various hepatocellular carcinoma (HCC) molecular subtypes, the analysis engine 115 may subject the features to one or more cluster analysis, fractal dimension measurements, and/or the like. For example, the presence of certain features, such as the presence of certain types of tissues, growth patterns, and cells within the image of the hepatocellular carcinoma (HCC) sample, may indicate that the hepatocellular carcinoma (HCC) sample is positive for one of a cholangio-like subtype, a hepatocyte-like subtype, and a progenitor-like subtype of hepatocellular carcinoma (HCC). In some cases, the molecular subtype that is present in the image of the hepatocellular carcinoma (HCC) sample may be identified, in whole or in part, by applying a feature identification model, an end-to-end model (e.g., ingesting whole slide images as input), a spatial signature model (e.g., a graph neural network (GNN) ingesting cell and tissue heat maps as input), and/or the like.

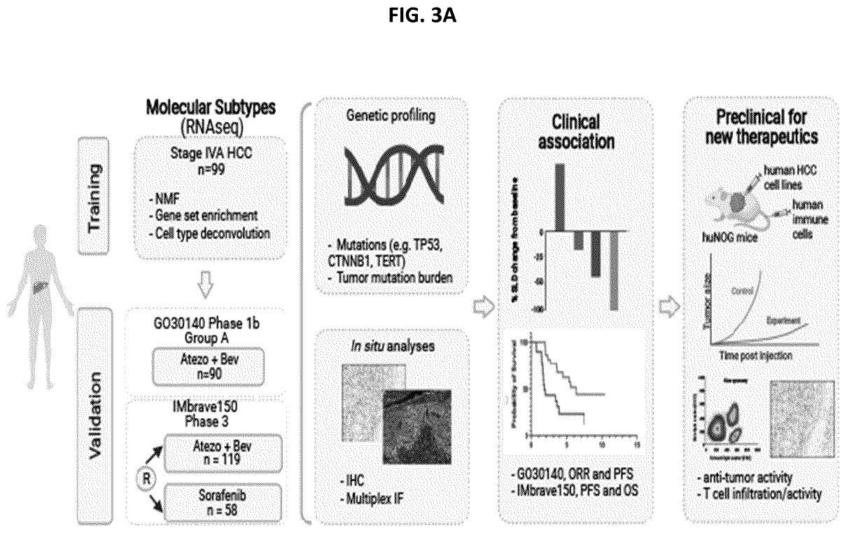

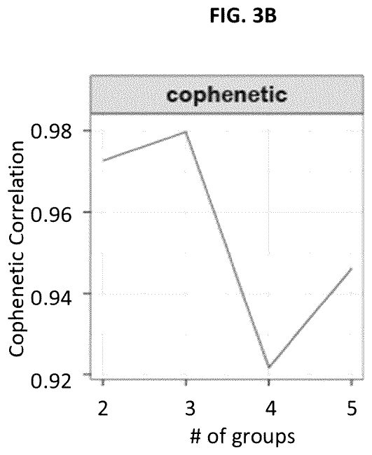

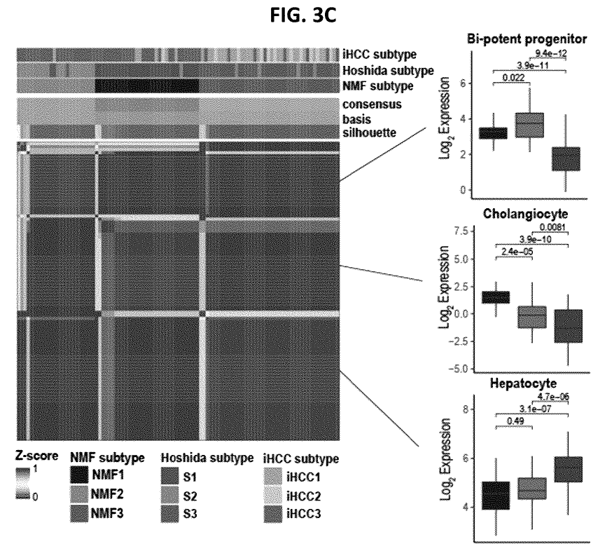

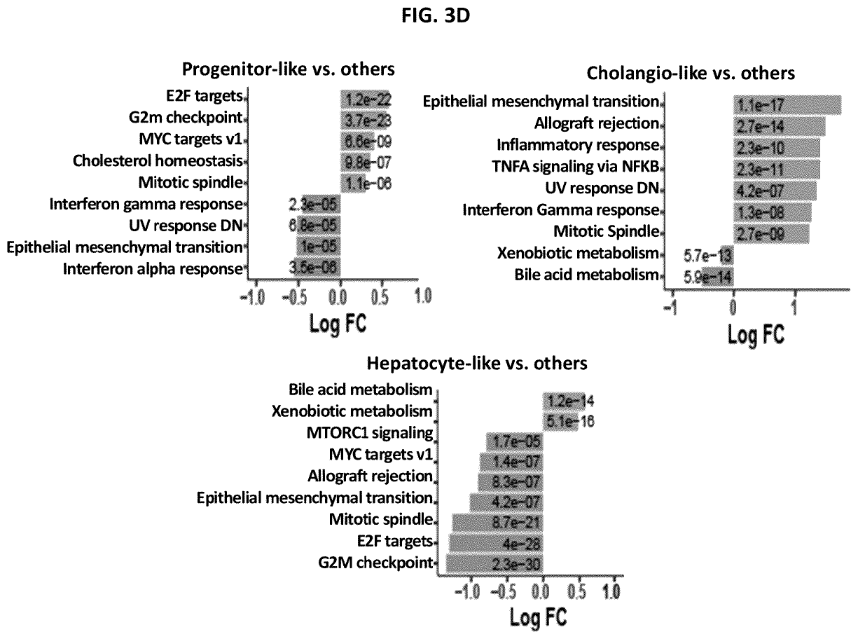

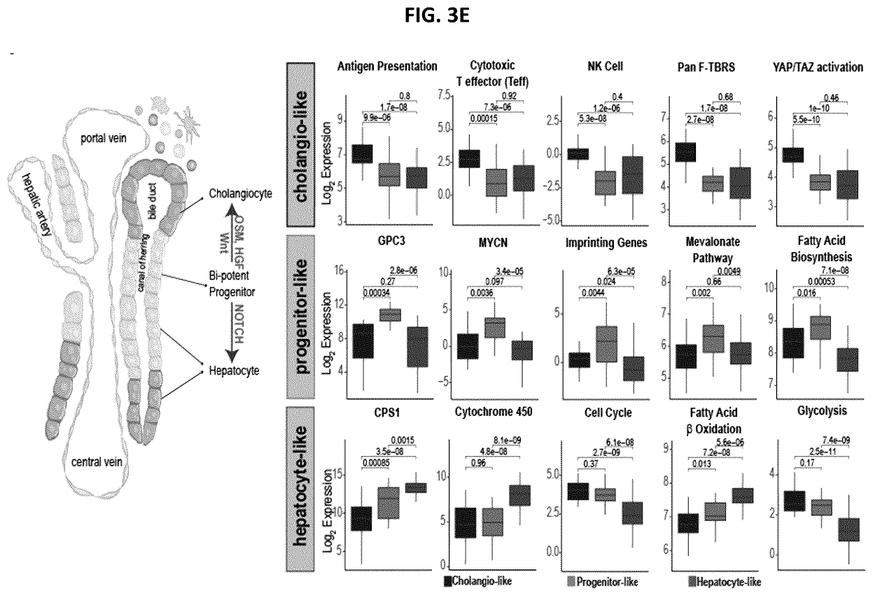

To further illustrate, FIG. 3A depicts an example in which the analysis engine 115 applies a non-negative matrix factorization (NMF) to the transcriptome data of various hepatocellular carcinoma tumor samples in order to determine the optimal number of subpopulations present therein. For example, with non-negative matrix factorization (NMF), the analysis engine 115 may iteratively select the most robust clustering pattern for the transcriptome data, including an optimal quantity of clusters (e.g., within a configurable range such as 2 to 8), such that the intra-cluster correlation of each resulting cluster is maximized. In the example shown in FIG. 3A, 99 stage IVA hepatocellular carcinoma resected tissue samples were used as a training set, and GO30140 Ph1b group A and IMbrave150 biomarker populations (BEP) were used as validation sets. FIG. 3B shows that the cluster analysis (e.g., the non-negative matrix factorization (NMF) may be applied to a portion of the training set exhibiting most highly variable genes. Moreover, as shown in FIG. 3B, three clusters, which is associated with the highest cophenetic correlation value (0.98), may be the optimal quantity of the subpopulations. FIG. 3C shows that these clusters (non-negative matrix factorization (NMF) subtypes in FIGS. 3A-3E) are highly overlapping with Hoshida's S1-S3 subtypes, respectively, identified by metadata consensus clustering analysis. Furthermore, FIG. 3C shows that when overlaid with previously identified metabolic subtypes iHCC1-3 identified through transcriptome and metabolic functional network analysis of the The Cancer Genome Atlas (TCGA) hepatocellular carcinoma set, iHCC3 overlaps with the first and second subtypes NMF1 and NMF2 identified through non-negative matrix factorization while the third non-negative matrix factorization subtype NMF3 is enriched for both iHCC1 and iHCC2 subtypes with similar metabolic programs that are distinctive from iHCC3.

Each non-negative matrix factorization (NMF) subtype identified through non-negative matrix factorization (NMF) may be associated with unique tumor intrinsic features and tumor microenvironment (TME) features. These relationships may be identified through an investigation of the association between molecular subtypes and liver epithelial lineages. For example, in a liver lobule, the bi-potent progenitor cells residing in the canal of herring can give rise to either cholangiocytes lining the bile duct near portal vein or mature hepatocytes by central vein, guided by molecular cues such as Notch signaling. The liver is therefore associated with three epithelial lineages, cholangiocytes, bi-potent progenitors, and hepatocytes, as indicated by the expression patterns of the corresponding cell lineage signatures. Remarkably, as shown in FIG. 3C, each of the subtypes identified through non-negative matrix factorization (NMF) has significantly higher expression of a particular cell lineage signature than the others. Accordingly, based on the dominant cell lineage signature expressed by each non-negative matrix factorization (NMF) subtype, the corresponding cluster is designated as a cholangio-like subtype, a progenitor-like subtype, and a hepatocyte-like subtype.

FIG. 3D and FIGS. 7A-7B depict the gene set enrichment analysis (GSEA) that is performed to discover the signaling pathways enriched in each subtype more comprehensively, we performed gene set enrichment analysis (GSEA). Meanwhile, FIGS. 7A-7B depicts the cell-type enrichment analysis (e.g., with xCell) that is performed on the RNA sequence data of the training set to discover the signaling pathways enriched in each subtype. The progenitor-like subtype shows strong cell-cycle transcriptional programs (G2M, E2F and MYC targets, mitotic spindle signature) but has relatively modest inflammation with lower interferon-alpha and -gamma immune response (FIG. 3D) and fewer conventional dendritic cells (aDC) and stromal cells (FIGS. 7A-7B) as compared to the other two subtypes. In contrast, the cholangio-like subtype features high inflammatory responses such as TNF-alpha and interferon-gamma and epithelial to mesenchymal transition (EMT), a pathway driven by TGFb (FIG. 3D). A deconvolution analysis with the cell-type enrichment analysis (e.g., with xCell) further confirmed that cholangio-like enriches for both innate and adaptive immune cell types, immunoscore, and the cholangiocyte-like feature (FIGS. 7A-7B). Lastly, the hepatocyte-like subtype features enrichment of signatures pertaining to hepatocyte functions including bile acid and xenobiotic metabolism pathways (FIG. 3C), and a cell type enrichment for hepatocytes (FIGS. 7A-7B). The hepatocyte-like subtype is also less proliferative, consistent with its overlap with Hoshida's non-proliferative S3 subtype.

In some example embodiments, the tumor intrinsic features and tumor microenvironment (TME) features of cell lineage associated subtypes may be further analyzed based on expression pattern among subtypes with gene expression signatures representing pathways identified by gene set enrichment analysis (GSEA) analysis, oncogenic signaling pathways involved in hepatocellular carcinoma initiation and prognosis, markers related to clinical outcome to atezolizumab plus bevacizumab, and liver metabolic pathways with gene expression signatures. Table 1 below enumerates examples of gene expression signatures.

TABLE 1

| Pathway | Genes | Sources and References |
|---|---|---|
| Bi-potent | DUSP9, IGSF1, RPL10A, E2F5, BUB1, DLGAP5, | Cairo, S., Cancer Cell 2008 |
| progenitor | NLE1 | |
| Imprinting | DLK1, ARID3A, H19, IGF2, ZIM2, PEG3, MEG8, | Upregulated in TCGA HCC vs cirrhosis and |
| gene | | associated with bad prognosis |
| Cholesterol | ATF5, GNAI1, LGMN, PLSCR1, PPARG, | Zhu, A., Nature Medicine, in revision |
| homeostasis | STARD4 | |
| Fatty acid | ACSL3, FASN, ACACA, ACLY, SCD | selected from MSigDB, |
| biosynthesis | | WP_FATTY_ACID_BIOSYNTHESIS gene |
| | | set |
| Mevalonate | ACAT2, FDPS, HMGCR, HMGCS1, MVD, MVK, | MSigDB, WP_MEVALONATE_PATHWAY |
| pathway | PMVK | gene set |
| Cholangiocyte | KRT7, KRT19, TFF2, SCGB3A1, FXYD2, DEFB1, | MacParland, S.A., Nature Communications, 2018 |
| | CD24, EPCAM | |
| YAP/TAZ | ARHGEF28, GADD45A, IRS1, EVA1A, NEDD4, | Pham T.H., Cancer Discovery, 2020 |
| activation | MGLL, DUSP1, GADD45B, STOM, ADM, DKK1, | |
| | ALCAM, LOC102723893, CPA4, ANKRD13A, | |
| | TBC1D23, SEMA3C, CCDC80, FSIP1, THBS1, | |
| | EFEMP1, COL12A1, LIMA1, LEPREL1, | |
| | EPB41L2, F3, AXL, UBASH3B, NABP1, CYR61, | |
| | FOSL1, OPHN1, ROR1, SMURF2, TGM2, | |
| | ANKRD1, PLK2, GAS6, C15orf52, AMOTL2, | |
| | CRIM1, STARD13, AKAP2, PALM2-AKAP2, | |
| | ARHGAP29, WWC2, PDLIM2, ANLN, CTNNAL1 | |
| Pan F-TBRS | ACTA2, ACTG2, ADAM12, ADAM19, AMIGO2, | Mariathasan, S., Nature 2018 |
| | CDKN2B, CNN1, COL4A1, CTGF, ELN, FSTL3, | |
| | HSPB1, IGFBP3, | |
| | SH3PXD2A, TAGLN, TGFBI, TNS1, TPM1 | |
| T effector | CXCL9, PRF1, GZMB | Zhu, A., Nature Medicine, in revision |
| (Teff) | | |
| Antigen | HLA-A, PSMB8, PSMB9, TAP1, TAP2 | Desbois M., Nature Communications, 2020 |
| Presentation | | |
| NK | KLRC4-KLRK1, KLRC4, CCL5 | Downregulated in TCGA HCC vs cirrhosis and |
| | | associated with good prognosis |
| Hepatocyte | BCHE, G6PC, ALDH6A1, RCAN1, AR, RPP25L, | MacParland, S.A., Nature Communications, 2018 |
| | HSD11B1, HAMP, GHR, APOM | |
| Cytochrome | CYP2B6, CYP1A2, CYP3A4, ADH1C, CYP2C8, | Downregulated in TCGA HCC vs cirrhosis and |
| P450 | CYP2C9, CYP2A6 | associated with good prognosis |
| Cell cycle | CDKN2C, E2F7, CDK2, CDK4, E2F1, EZH2, | Upregulated in TCGA HCC vs cirrhosis and |
| | E2F8, BIRC5, MKI67, CCNB2, CDK1, CCNB1, | associated with bad prognosis |
| | CCNA2, CCNE1, CCNE2 | |
| Glycolysis | DPYSL4, HOMER1, ABCB6, CENPA, CDK1, | Jiang, L., Glycolysis gene expression profilings |
| | STMN1 | screen for prognostic risk signature of |
| | | hepatocellular carcinoma Aging (Albany NY), 2019 |
| Fatty acid | CPT1A, EHHADH, HADHA, ACADM, ACAT1, | selected from MigDB, |
| oxidation | ACOX1, ACOX2, ACOX3 | Hallmark_Fatty_Acid_Beta_Oxidation gene set |

In addition to confirming the inflammatory immune microenvironment of the cholangio-like subtype with high expression of CD274 (PD-L1 mRNA), T effector, antigen presentation and NK cell signatures, the cholangio-like subtype is also determined to exhibit a high YAP/TAZ activation and fibroblast TGFb response signature (F-TBRS) (FIG. 3E). Interestingly, the progenitor-like subtype has re-expression of oncofetal genes including GPC3 and AFP and many imprinted genes such as DLKT, IGF2 and ZIM2 (Table 1), which indicates a less differentiated state. High expression of MYCN is consistent with high cell cycle activity identified for progenitor-like by GSEA (FIG. 3D). The hepatocyte-like subtype was characterized by significantly higher expression of pathways and signatures representing key biosynthesis and metabolic function of well differentiated hepatocytes such as cytochrome 450s (CYPs) which is related to detoxification of xenobiotics and cellular metabolism. This subtype also had lower expression of cell cycle genes compared to the cholangio-like and progenitor-like subtypes (FIG. 3E).

In some example embodiments, subtype specific metabolic pathways may be analyzed based on expression patterns of MSigDB (http://www.gsea-msigdb.org/gsea/msigdb/collections.jsp) signatures of multiple metabolic programs. Similar to metabolic subtype iHCC3, cholangio-like and progenitor-like subtypes have more influx in glycolysis and fatty acid biosynthesis (FAB) but less TCA cycle and fatty acid oxidation (FAO) compared to hepatocyte-like with similar metabolic reliance as iHCCT-2 subtypes (FIG. 3E).

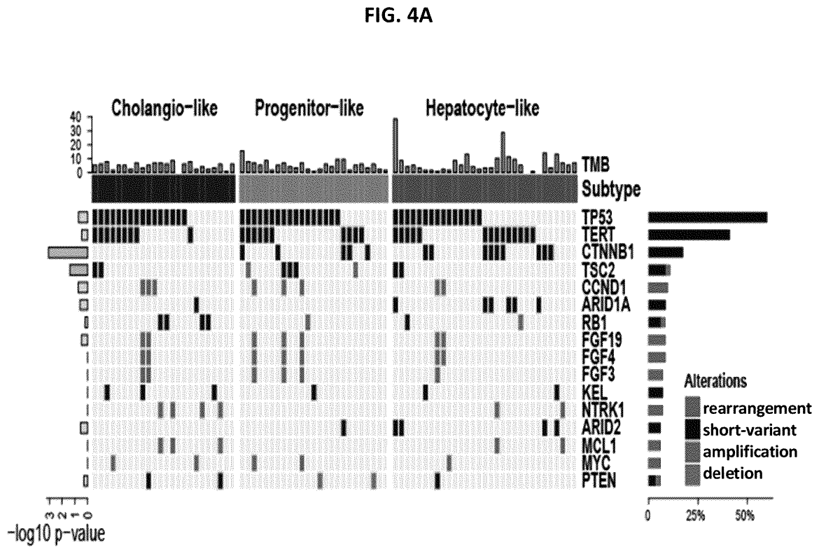

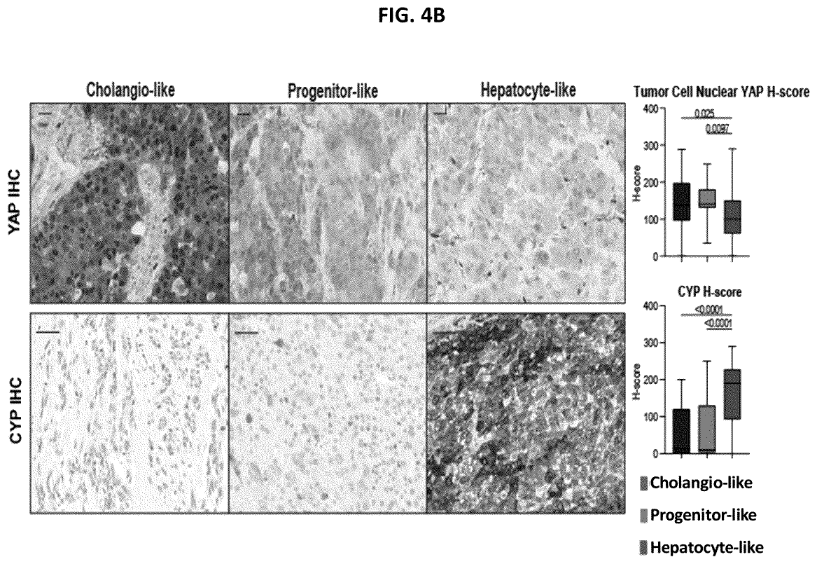

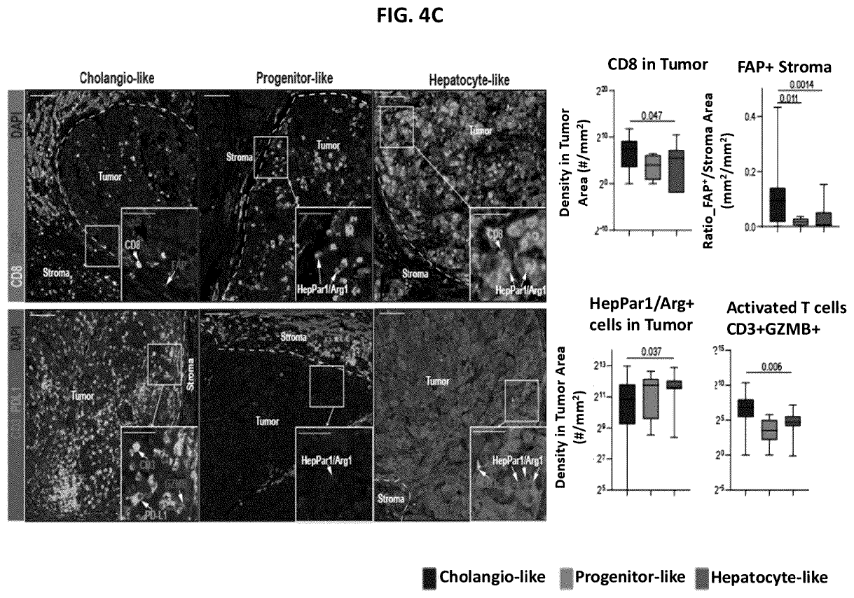

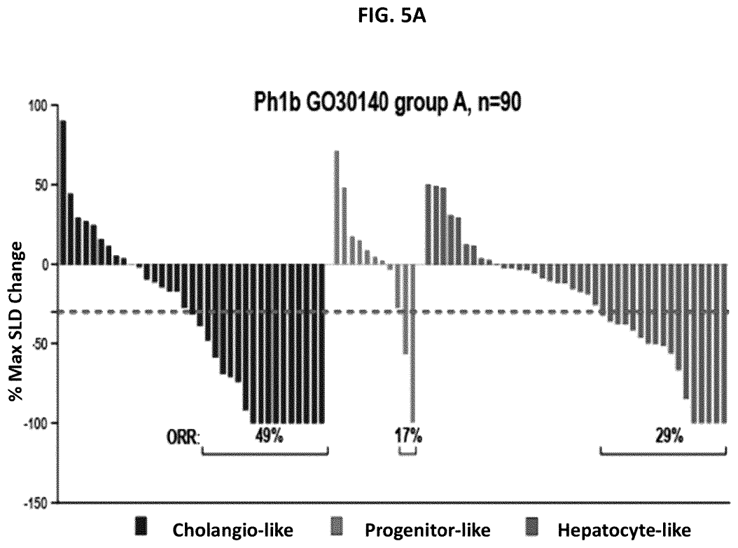

Referring now to FIG. 4A, genetic features associated with the hepatocellular carcinoma subtypes were evaluated with FoundationOne panel profiling of the 99 tumor tissues in the training set. No significant difference was observed among subtypes in terms of tumor mutation burden (TMB) or genomic instability such as loss of heterozygosity (LOH) levels. On the other hand, the mutation landscape of the training set confirmed frequent disease-associated somatic mutations in hepatocellular carcinoma, including TP53 (58%), TERT promoter (39%) and CTNNB1 mutations (17%). Importantly, CTNNB1 mutations were mostly restricted in the hepatocyte-like subtype (12/14, 86%, P value of association with subtypes is 0.0026); whereas the TP53 alterations were more prevalent in cholangio-like and progenitor-like (P=0.022) and mutually exclusive with CTNNB1 (Fisher Exact test, P=0.019) alterations (FIG. 4A). Together, the subtype-specific genetic profile further highlighted the importance of tumor cell-intrinsic features in shaping the distinct oncogenic machinery of the hepatocellular carcinoma subtypes.

One or more in situ assays validating subtype specific molecular features are performed to further distinguish the effects of tumor cell-intrinsic features and tumor microenvironment (TME) features in shaping the hepatocellular carcinoma subtypes. FIG. 4B shows that genes downstream of YES-associated protein (YAP) activation are highly expressed in the cholangio-like subtype. YES-associated protein (YAP) activation starts from the translocation of unphosphorylated YES-associated protein (YAP) into the nucleus, then the YES-associated protein (YAP) forms a transcription factor complex with TAZ and TEAD to promote cell proliferation, dedifferentiation and trans-differentiation. Therefore, YES-associated protein (YAP) activation may be evaluated by performing nuclear YES-associated protein (YAP) immunohistochemistry (IHC) analysis on 94 FFPE sections from the training set to evaluate YAP activation. Representative images and a box plot summarizing H-scores of nuclear tumor cell YAP staining are shown in FIG. 4B. As shown in FIG. 4B, the H-scores of cholangio-like and progenitor-like tumors are significantly higher than those of hepatocyte-like (P=0.025 and 0.010 for cholangio-like vs hepatocyte-like and progenitor-like vs hepatocyte-like, respectively).

Next, the enrichment of CYPs expression in hepatocyte-like subtype may be confirmed with an immunohistochemistry (IHC) assay detecting CYP2C8, CYP2C9, CYP2C18 and CYP2C19. Cytoplasmic CYP expression of tumor cells in 90 tissue sections from the training set was measured with H-scores. Consistent with the RNA sequences, CYP expression is the highest in hepatocyte-like hepatocellular carcinoma (FIG. 4B) with a mean tumor cell H-score of 163 (0-290) (FIG. 4B). Almost the entirety of assayed hepatocyte-like hepatocellular carcinoma (e.g., 44 of 45 (98%)) had CYP expression (H-scores>0). Contrastingly, only 64% (18/28) of cholangio-like and 82% (14/17) of progenitor-like subtype tumor cells had detectable CYP (FIG. 4B). The CYP H-scores of hepatocyte-like were dramatically higher than those of other subtypes (p<0.0001 for both cholangio-like vs hepatocyte-like and progenitor-like vs hepatocyte-like) (FIG. 4B).

Two multiplex immunofluorescence (IF) panels was developed to survey tumor-stromal-immune contexture (Panel 1) and T-cell functionality (Panel 2) features of the cholangio-like, progenitor-like, and hepatocyte-like subtypes in 64 baseline biopsies from GO30140 group A. A digital pathology scoring algorithm was developed to measure density of cells with certain phenotypes (single or multiple markers) in epithelial tumor bed, stromal area or total tumor areas for each panel. An antibody cocktail against Hepatocyte Paraffin 1 (HepPar1) and Arginase 1 (Arg1) was incorporated in both panels to mark hepatocellular carcinoma tumor cells. FIG. 4C shows representative images of Panel 1 staining of all 5-markers in different molecular subtypes and summarizing digital scores of key tumor-stromal-immune-context features, respectively. Similarly, representative images and digital scores of key features of Panel 2 in molecular subtypes are also shown FIG. 4C. As shown in FIG. 4C, the density of HepPar1/Arg1⁺, cells in tumor area (calculated by number (#) of HepPar1/Arg1⁺ in mm² of tumor area) is much higher in hepatocyte-like than cholangio-like subtype. Progenitor-like tumor cells have an intermediate average density of HepPar1/Arg1⁺ cells (FIGS. 5E-5H) and a variable expression of HepPar1/Arg1. In addition, expression of two known cholangiocyte related genes, SOX9 and KRT19, is the highest in cholangio-like subtypes across our data sets (FIG. 10). Interestingly the adjacent normal liver tissues (if any) showed stronger HepPar1/Arg1 staining than tumor cells even in hepatocyte-like hepatocellular carcinoma (FIGS. 5E and 5G).

Panel 1 of multiplex immunohistochemistry (IHC) was designed to survey CD8 T cell infiltration, antigen presentation, TGFβ stimulated reactive stroma and angiogenesis in TME measured by antibodies against CD8, MHC-I, FAP and CD31, respectively. Consistent with transcriptomic activation of effector T-cells (T_{eff}) and TGFβ signaling observed in the cholangio-like tumors (FIG. 3B), CD8 density in tumor area and % FAP+ area in stroma area are also significantly higher (not significant) in this subtype. Panel 2 contains antibodies against PD1, GZMB, CD3 and PDL1 for surveying T cell functional states (FIGS. 5G and 5H). As shown in FIG. 4B, PDL1+ cells, CD3 T cells within the tumor bed (epitumor), activated (CD3+GZMB+) and exhausted T cells (CD3+PD1+) in the tumor region are the highest in the cholangio-like subtype.

In some example embodiments, the hepatocellular carcinoma (HCC) molecular subtypes identified through the aforementioned cluster analysis (e.g., non-negative matrix factorization (NMF) and/or the like) may be validated in independent cohorts by applying a classifier (e.g., a random forest classifier) trained on a gene set from the RNA sequence data in the training set. The classifier predicted the same three hepatocellular carcinoma subtypes from the tumor samples in the GO30140 Phase 1b group A (n=90) and the IMbrave150 Phase 3 hepatocellular carcinoma clinical trials (n=178). Moreover, the prevalence of each subtype in the training and the two testing cohorts was also largely consistent (one-way ANOVA P-value>0.9999) (FIGS. 8A-8C), confirming the robustness of these subtypes. Consistent with high immune presence, a majority (94% in GO30140 group A and 79% in IMbrave150) of the cholangio-like samples have either immune cell (IC) or tumor cells (TC) with PD-L1 expression (≥1%). Furthermore, the frequencies of small nucleotide variations (SNVs) and small insertions and deletions (Indels) of CTNNB1 and TP53 genes (hcc biomarker manuscript) were also similar among three data sets and that of the TCGA hepatocellular carcinoma data set⁹ (might need some statistics to substantiate this). Comparing known demographic prognostic factors distribution among subtypes across datasets showed the hepatocyte-like had the highest proportion of well-differentiated tumors. The results of this comparison are shown in Tables 2-4 below.

Table 2 below depicts the demographic characteristics of molecular subtypes within the training set.

TABLE 2
Training (n = 99)

| | Cholangio- | | Progenitor- | | Hepatocyte- | |
|---|---|---|---|---|---|---|
| | like | | like | | like | |
| Prevalence, n (%) | 33 | (33) | 20 | (20) | 46 | (46) |
| Age, n (%) | | | | | | |
| <65 yr | 31 | (94) | 17 | (85) | 37 | (80) |
| >=65 yr | 2 | (6) | 3 | (15) | 9 | (20) |
| Sex, n (%) | | | | | | |
| Male | 26 | (79) | 14 | (70) | 37 | (80) |
| Female | 7 | (21) | 6 | (30) | 9 | (20) |
| Etiology, n(%) | | | | | | |
| HBV | 32 | (97) | 19 | (95) | 46 | (100) |
| HCV | 1 | (3) | 1 | (5) | 0 | (0) |
| TNM stage, n (%) | | | | | | |
| T2 N1 M0 | 17 | (52) | 10 | (50) | 31 | (67) |
| T3 N1 M0 | 16 | (48) | 10 | (50) | 15 | (33) |
| Disease stage, n (%) | | | | | | |
| IVA | 33 | (100) | 20 | (100) | 46 | (100) |
| Histology Grade, n (%) | | | | | | |
| Well Diffrentiated | 1 | (3) | 2 | (10) | 10 | (22) |
| Moderately Differentiated | 25 | (76) | 10 | (50) | 33 | (72) |
| Poorly Differentiated | 7 | (21) | 8 | (40) | 3 | (7) |

Table 3 below depicts the emographic characteristics and know hepatocellular carcinoma prognostic factors among molecular subtypes in Ph1b GO30140 Arm A biomarker evaluable population (BEP).

TABLE 3
GO30140 Arm A BEP (n = 90)

| | Cholangio- | | Progenitor- | | Hepatocyte- | |
|---|---|---|---|---|---|---|
| | like | | like | | like | |
| Prevalence, n (%) | 37 | (41) | 12 | (13) | 41 | (46) |
| Age Group, n (%) | | | | | | |
| <65 yr | 27 | (73) | 9 | (75) | 22 | (54) |
| >=66 yr | 10 | (27) | 3 | (25) | 19 | (46) |
| Sex, n (%) | | | | | | |
| Male | 33 | (89) | 5 | (42) | 37 | (90) |
| Female | 4 | (11) | 7 | (58) | 4 | (10) |
| Etiology, n (%) | | | | | | |
| HBV | 24 | (65) | 6 | (50) | 19 | (46) |
| HCV | 7 | (19) | 3 | (25) | 12 | (29) |
| Non-viral | 6 | (16) | 3 | (25) | 10 | (24) |
| Stage at Time of | | | | | | |
| Study Entry, n (%) | | | | | | |
| II | 2 | (5) | 0 | (0) | 3 | (7) |
| III | 7 | (19) | 2 | (17) | 9 | (22) |
| IV | 28 | (76) | 10 | (83) | 29 | (71) |
| BCLC Staging Class | | | | | | |
| at Study Entry, n (%) | | | | | | |
| Stage B | 5 | (14) | 0 | (0) | 5 | (12) |
| Stage C | 32 | (86) | 12 | (100) | 36 | (88) |
| Histology Grade, n (%) | n = 20 | | n = 4 | | n = 23 | |
| Well Differentiated | 3 | (15) | 0 | (0) | 7 | (30) |
| Moderately Differentiated | 7 | (35) | 2 | (50) | 13 | (57) |
| Poorly Differentiated | 10 | (50) | 2 | (50) | 3 | (13) |
| Child Pugh Class, n (%) | | | | | | |
| A5/A6 | 37 | (100) | 11 | (92) | 38 | (93) |
| B7 | 0 | (0) | 1 | (8) | 3 | (7) |
| Tumor Occupies >50% | | | | | | |
| of Liver, n (%) | | | | | | |
| Yes | 1 | (3) | 1 | (8) | 7 | (17) |
| No | 36 | (97) | 11 | (92) | 34 | (83) |
| Main Portal Vein | | | | | | |
| Invasion (Vp4), n (%) | | | | | | |
| Yes | 3 | (8) | 1 | (8) | 6 | (15) |
| N0 | 34 | (92) | 11 | (92) | 35 | (85) |
| Macro Vascular | | | | | | |
| Invasion (MVI), n (%) | | | | | | |
| Yes | 20 | (54) | 6 | (60) | 23 | (56) |
| No | 17 | (46) | 6 | (50) | 18 | (44) |
| Extrahepatic | | | | | | |
| Spread (EHS), n (%) | | | | | | |
| Yes | 28 | (76) | 10 | (83) | 29 | (71) |
| No | 9 | (24) | 2 | (17) | 12 | (29) |
| Baseline Serum | n = 36 | | n = 11 | | n = 37 | |
| AFP Category, n (%) | | | | | | |
| 0-<400 ng/ml | 24 | (67) | 5 | (45) | 22 | (59) |
| >=400 ng/ml | 12 | (33) | 6 | (55) | 15 | (41) |
| ECOG, n (%) | | | | | | |
| 0 | 22 | (59) | 4 | (33) | 24 | (59) |
| 1 | 15 | (41) | 8 | (67) | 17 | (41) |
| Prior Local Therapy, n (%) | | | | | | |
| Yes | 26 | (70) | 6 | (50) | 28 | (68) |
| No | 11 | (30) | 6 | (50) | 13 | (32) |

Table 4 below depicts the demographic characteristics and known hepatocellular carcinoma prognostic factors among molecular subtypes in BEP of Ph III IMBrave 150.

IMBrave 150 BEP (n = 178)

| | Cholangio- | | Progenitor- | | Hepatocyte- | |
|---|---|---|---|---|---|---|
| | like | | like | | like | |
| Prevalence, n (%) | 50 | (26) | 28 | (16) | 100 | (56) |
| Age Group, n (%) | | | | | | |
| <65 yr | 29 | (58) | 16 | (57) | 70 | (70) |
| >=65 yr | 21 | (42) | 12 | (43) | 30 | (30) |
| Sex, n (%) | | | | | | |
| Male | 44 | (88) | 18 | (64) | 80 | (80) |
| Female | 6 | (12) | 10 | (38) | 20 | (20) |
| Etiology, n (%) | | | | | | |
| HBV | 23 | (46) | 15 | (54) | 31 | (31) |
| HCV | 9 | (18) | 8 | (29) | 22 | (22) |
| Non-viral | 18 | (36) | 5 | (18) | 47 | (47) |
| Stage at Time of | | | | | | |
| Study Entry, n (%) | | | | | | |
| B/II | 7 | (14) | 5 | (18) | 19 | (19) |
| III | 8 | (16) | 3 | (11) | 31 | (31) |
| IV | 35 | (70) | 20 | (71) | 50 | (50) |
| BCLC Staging Class | | | | | | |
| at Study Entry, n (%) | | | | | | |
| Stage A | 0 | (0) | 0 | (0) | 5 | (5) |
| Stage B | 8 | (16) | 2 | (7) | 16 | (16) |
| Stage C | 42 | (84) | 26 | (93) | 79 | (79) |
| Histology Grade, n (%) | n = 31 | | n = 19 | | n = 70 | |
| Well Differentiated | 3 | (10) | 1 | (5) | 28 | (28) |
| Moderately Differentiated | 14 | (45) | 10 | (53) | 35 | (35) |
| Poorly Differentiated | 14 | (45) | 8 | (42) | 7 | (7) |
| Child Pugh Class, n (%) | n = 49 | | n = 28 | | n = 99 | |
| A5/A6 | 49 | (100) | 28 | (100) | 99 | (100) |
| Tumor Occupies >50% | | | | | | |
| of Liver, n (%) | | | | | | |
| Yes | 2 | (4) | 1 | (4) | 6 | (6) |
| No | 48 | (96) | 27 | (96) | 94 | (94) |
| Main Portal Vein | | | | | | |
| Invasion (Vp4), n (%) | | | | | | |
| Yes | 11 | (22) | 0 | (0) | 9 | (9) |
| No | 39 | (78) | 28 | (100) | 91 | (91) |
| Macro Vascular | | | | | | |
| Invasion (MVI), n (%) | | | | | | |
| Yes | 21 | (42) | 8 | (29) | 36 | (36) |
| No | 29 | (58) | 20 | (71) | 64 | (64) |
| Extrahepatic Spread | | | | | | |
| (EHS), n (%) | | | | | | |
| Yes | 36 | (72) | 20 | (71) | 50 | (50) |
| No | 14 | (28) | 8 | (29) | 50 | (50) |
| Baseline Serum | | | | | | |
| AFP Category, n (%) | | | | | | |
| 0-<400 ng/ml | 29 | (58) | 10 | (36) | 79 | (79) |
| >=400 ng/ml | 21 | (42) | 18 | (64) | 21 | (21) |
| ECOG, n (%) | | | | | | |
| 0 | 33 | (66) | 20 | (71) | 64 | (64) |
| 1 | 17 | (34) | 8 | (29) | 36 | (36) |
| Prior Local Therapy, n (%) | | | | | | |
| Yes | 17 | (34) | 18 | (57) | 47 | (47) |
| No | 33 | (66) | 12 | (43) | 53 | (53) |
| Actual Treatment, n (%) | | | | | | |
| Atezo + Bev | 34 | (68) | 20 | (71) | 65 | (65) |
| Sorafenib | 15 | (30) | 8 | (29) | 35 | (35) |
| Untreated | 1 | (2) | 0 | (0) | 0 | (0) |

In some example embodiments, effector T-cell (T_{eff}) and CD274 expression may be associated with patient response and/or pathological response (e.g., major pathological response (MPR)) to combination immunotherapy (e.g., atezolizumab (anti-PD-L1) and bevacizumab (anti-VEGF) combination therapy) whereas the ratio of regulator T-cells (T_{reg}) and effector T-cells (T_{eff}), GPC3 expression, and AFP expression are associated with less clinical benefit from combination immunotherapy. As shown in FIG. 2E, these genomic associates have differential expression among the three hepatocellular carcinoma (HCC) molecular subtypes. Indeed, with the highest expression of effector T-cells (T_{eff}) and CD274 and immune cell infiltration, cholangio-like subtype patients in GO30140 group A had the best objective response rate (ORR) by independent review forum (IRF, the primary clinical endpoint of group A) (49%, 18/37) among subtypes, a much higher rate than that of intent-to-treat (ITT) population of group A patients (36%). By contrast, the progenitor-like subtype showed less than half of the ORR (17%, 2/12) of intent-to-treat (ITT) population (FIG. 5A), possibly due to the upregulation of GPC3 and AFP, genes associated with less clinical activity of atezolizumab (anti-PD-L1) and bevacizumab (anti-VEGF) combination therapy (HCC primary biomarker manuscript). The median progression free survival rate (mPFS) of the cholangio-like subtype of patients (9.5 months) is 5 times longer than that of progenitor-like patients (1.9 months) (FIG. 5B). The hepatocyte-like subtype patients had an intermediate objective response rate (ORR) of 29% (12/41) and medium progression free survival rate (mPFS) (9.9 months) similar to cholangio-like patients.

The association of molecular subtypes with response and resistance to combination immunotherapy, such as atezolizumab (anti-PD-L1) and bevacizumab (anti-VEGF) combination therapy, was further validated in the PhIII randomized trial IMbrave150. Notably, FIG. 6C shows significantly longer overall survival (OS) benefit of atezolizumab (anti-PD-L1) and bevacizumab (anti-VEGF) combination therapy over sorafenib in both cholangio-like (HR, CI=0.37, 0.15-0.91; P=0.039) and hepatocyte-like (HR, CI=0.41, 0.18-0.91; P=0.029) subtypes. Contrastingly, patients exhibiting the progenitor-like subtype derived less benefit in terms of progression free survival (PFS) and overall survival (OS) from the atezolizumab (anti-PD-L1) and bevacizumab (anti-VEGF) combination therapy over sorafenib (FIG. 6C). Both high expression of oncofetal protein GPC3 and immune desert-like phenotype in the progenitor-like group might contribute to the resistance to atezolizumab (anti-PD-L1) and bevacizumab (anti-VEGF) combination therapy. Interestingly, a recent single-cell RNA sequence study has revealed that oncofetal features of hepatocellular carcinoma orchestrated an immunosuppressive tumor microenvironment through co-localization and interaction of fetal-like PLVAP⁺ endothelial cells and FOLR2⁺ tumor associated macrophages with TIGIT⁺ Tregs. Consistently, significantly higher regular T-cell (T_{reg}) and effector T-cell (T_{eff}) ratios were observed in the progenitor-like subtype in GO30140 group A and a similar trend in other two data sets (FIG. 11).

In some example embodiments, subtype specific resistance to combination immunotherapy, such as the resistance to atezolizumab (anti-PD-L1) and bevacizumab (anti-VEGF) combination therapy associated with the progenitor-like subtype, may be overcome by formulating subtype-specific treatment strategies to target subtype-specific vulnerabilities. For example, the addition of an anti-GPC3/anti-CD3 bispecific antibody (ERY974) may overcome subtype specific resistance to combination immunotherapy by eliciting a strong anti-tumor activity through induction of T cell infiltration and activation in GPC3 expressing progenitor-like tumors. To evaluate GPC3 as a target for the progenitor-like subtype, the anti-GPC3/anti-CD3 bispecific antibody (ERY974) was assessed in humanized NOG (huNOG) xenograft models with generated with hepatocellular carcinoma cell lines. To select hepatocellular carcinoma cell lines for the models, 16 lines were classified based on a hierarchical clustering of 288 classifier genes (FIG. 12). Among them, huH-1 and huH-7 shared multiple molecular features as progenitor-like subtypes including the high expression of GPC3¹⁵(FIG. 6A). FIG. 6B shows confirmation, through fluorescence-activated cell sorting (FACS), of high cell surface expression of the GPC3 protein. Antibody binding capacity of ERY974 in huh-1 and huh-7 is 5.94×10⁴/cell, and 4.85×10⁴/cell, respectively. By contrast, FIG. 6C GPC3 surface expression is previously under detection limit in SK-HEP-1. In vitro T-cell dependent cytotoxicity (TDCC) assay further confirmed that the binding of ERY974 results in strong T cell mediated cell killing in huh-1 and huh-7 lines but not in SK-HEP-1 line (FIG. 6D).

The anti-tumor activity and pharmacodynamics of target inhibition of ERY974 were also examined in vivo in huh-1 and huh-7 xenograft models. As the anti-human CD3 arm of ERY974 does not cross-react with murine CD3, to supply human T cells to huh-1 huNOG models, human CD34+ stem cells were injected intravenously. For the huh-7 model, T cells from healthy donors were ex-vivo expanded and activated before their administration to circulation. As shown in FIG. 6E, ERY974 showed a strong anti-tumor activity in both progenitor-like huh-land huh-7 huNOG models. Tumor growth inhibition index (TGI) for huh-1 models is 109% and is 105% for huh-7 models. On the other hand, ERY974 showed no anti-tumor activity in cholangio-like SK-HEP-1 model.

To determine if the anti-tumor efficacy of ERY974 in progenitor-like xenograft models are related to recruitment and activity of T cells in the tumor, flow cytometry, CD3 and CD8 immunohistochemistry (IH) analysis, and nanostring gene expression were performed to examine T cell tumor infiltration and activation in huh-1 tumors after 3 days of 5 mg/kg ERY974 (FIG. 6F). Fluorescence-activated cell sorting (FACS) analysis showed drastically increased CD4 and CD8 T cell infiltration into the tumor. In addition, expression of GZMB, PD1 and IFNγ, markers of T cell activation, also dramatically increased (FIG. 4G). As shown in FIG. 6H and Table 5 below, histopathological evaluation of CD3 and CD8 IHC images further validated T cell and myeloid cell infiltration as well as tumor cell killing upon ERY974 treatment. Furthermore, the activation of infiltrating immune cells introduced by ERY974 is evident from elevated expression of signature genes representing T cells in different functional states including effector and exhausted T cells (FIG. 6I).

Table 5 below depicts a histopathological analysis of sections of huh-1/huNOG tumor.

TABLE 5

| | Model | | | | | | | | | | | | | | | |
|---|---|---|---|---|---|---|---|---|---|---|---|---|---|---|---|---|
| | huH1/huNOG | | | | | | | | | | | | | | | |
| | Sampling time | | | | | | | | | | | | | | | |
| | Day 3 | | | | | | | | | | | | | | | |
| | Treatment | | | | | | | | | | | | | | | |
| | vehicle | | | | | | | | ERY974 | | | | | | | |
| Slide No | G1-1 | G1-2 | G1-3 | G1-4 | G1-5 | G1-6 | G1-7 | G1-8 | G2-1 | G2-2 | G2-3 | G2-4 | G2-5 | G2-6 | G2-7 | G2-8 |
| Inflammatory cells | | | | | | | | | | | | | | | | |
| Granulocyte* | | | | | | | | | 2 | 1 | 2 | 2 | 2 | 2 | 2 | 2 |
| Intratumor/central | — | — | — | — | — | — | — | — | 2 | 2 | 2 | 2 | 2 | 2 | 2 | 2 |
| Host-tumor interface | — | — | — | — | — | — | — | — | 1 | 1 | 1 | 1 | 1 | 1 | 1 | 2 |
| Outside the tumor | — | — | — | — | — | — | — | — | | | | | | | | |
| Mononuclear/Lymphoid cell* | | | | | | | | | | | | | | | | |
| Intratumor/central | — | 2 | — | — | — | — | — | 2 | 1 | 1 | 2 | 2 | 2 | 2 | 2 | 2 |
| Host-tumor interface | — | 1 | 1 | 1 | 1 | 1 | — | 1 | 2 | 2 | 2 | 2 | 2 | 2 | 2 | 2 |
| Outside the tumor | — | — | — | — | — | — | — | — | 1 | 2 | 1 | 2 | 2 | 2 | 2 | 2 |
| Tumor cells** | | | | | | | | | | | | | | | | |
| Cell death/Necrosis area | | | | | | | | | | | | | | | | |
| Intratumor/central | 1 | 1 | 1 | 1 | 1 | 1 | 1 | 1 | 3 | 3 | 3 | 3 | 2 | 2 | 3 | 3 |
| Host-tumor interface | 1 | 1 | 1 | 1 | 1 | 1 | 1 | 1 | 3 | 3 | 3 | 3 | 2 | 2 | 3 | 3 |
| Focal necrosis/degeneration | — | 2 | — | — | 1 | — | — | 2 | 3 | 3 | 3 | 3 | 2 | — | 3 | 3 |
| Fibrosis/granular tissue, inter | — | 1 | — | 1 | 1 | 1 | — | 1 | — | 1 | 1 | 1 | — | — | 1 | 2 |
| tumor | | | | | | | | | | | | | | | | |
| Fibrosis, Host-tumor interface | — | — | — | — | — | — | — | 1 | — | 2 | 2 | 2 | 2 | 1 | 2 | 2 |
| Tumor cells morphology** | | | | | | | | | | | | | | | | |
| Trabecular pattern including | | | | | | | | | | | | | | | | |
| the following cell type | | | | | | | | | | | | | | | | |
| Clear cell/steatohepatitic | 1 | 1 | 1 | 1 | 1 | 1 | 1 | 1 | 1 | — | — | — | — | — | — | — |
| Pseudoglandular | 2 | 2 | — | 1 | — | 2 | 1 | 2 | — | — | — | — | — | — | — | — |
| Mitosis | 2 | 2 | 2 | 2 | 2 | 2 | 2 | 2 | — | — | — | — | — | — | — | 1fo |
| Congestion/hemorrhage, intratumor | 2 | 2 | 2 | 2 | 2 | 2 | 1 | 2 | 3 | 3 | 1 | 2 | 2 | — | 2 | — |
| Myxomatous/edematous area, | P | P | — | P | — | — | — | P | — | P | P | P | — | P | P | P |
| intratum | | | | | | | | | | | | | | | | |
| Immunohistochemistry*** | | | | | | | | | | | | | | | | |
| CD3 | | | | | | | | | | | | | | | | |
| Intratumor/central | 1 s | 2 | 1 fo | 1 fo | 1 s | 1 s | 1 s | 2 | 3 | 3 | 3 | 3 | 3 | 3 | 3 | 3 |
| Host-tumor interface | 1 fo | 1 fo | 1 fo | 1 fo | 1 fo | 1 fo | 1 s | 1 | 3 | 3 | 3 | 2 | 3 | 3 | 3 | 3 |
| Outside the tumor | — | 1 fo | — | — | — | — | — | 1 fo | 1 | 2 | 2 | 1 | 1 | 2 | 1 | 2 |
| CD8 | | | | | | | | | | | | | | | | |
| Intratumor/central | 1 s | 2 | 1 fo | 1 s | 1 s | 1 fo | 1 s | 2 | 3 | 3 | 3 | 3 | 3 | 3 | 3 | 3 |
| Host-tumor interface | 1 fo | 1 fo | 1 fo | 1 fo | 1 s | 1 fo | 1 s | 1 | 3 | 3 | 3 | 3 | 3 | 3 | 3 | 3 |
| Outside the tumor | — | 1 fo | 1 s | — | — | — | — | 1 s | 1 | 1 | 2 | 1 | 1 | 1 | 1 | 1 |

*—, not ovserved; 1, scattered or rare; 2, diffuse, low density or focal; 3, diffuse, high density
**—, not observed; 1, slight; 2, moderate; 3, severe fo; focal, p; present
***—, not observed; 1, slight; 2, moderate; 3, severe fo; focal, s; single cell

As noted, in some example embodiments, a cluster analysis may be applied to transcriptome data to identify three distinctive hepatocellular carcinoma (HCC) molecular subtypes with a strong linkage to liver epithelial cell lineage as well as unique tumor intrinsic features and tumor microenvironment (TME) features. The three molecular subtypes were faithfully recapitulated in samples from Ph1b GO30140 and Ph 3 IMbrave150 trials classified with a classifier (e.g., an ensemble learning model such as a random forest classifier). The three molecular subtypes exhibited a differential response to combination immunotherapy, such as atezolizumab (anti-PD-L1) plus bevacizumab (anti-VEGF) combination therapy, with the cholangio-like subtype having the best response while the progenitor-like subtype showing the most resistance. To overcome the subtype-specific resistance associated with the progenitor-like subtype, combination immunotherapy may be administered along with GPC3. In particular, the anti GPC3/CD3 bi-specific antibody (ERY974) induced T cell infiltration and activation to elicit a strong anti-tumor activity in humanized hepatocellular carcinoma xenograft mouse models.

The association between hepatocellular carcinoma (HCC) molecular subtype and liver cell lineage is shown to be evident not only at transcription level but is also validated by in situ protein expression of HepPar1 and Arg1, two mature hepatocyte markers commonly used for differentiating hepatocellular carcinoma from liver metastases from other organs. Hepatocyte-like has the highest expression of mature hepatocyte marker genes and HepPar1/Arg1 among the 3 subtypes, suggesting this subtype retains most of the molecular features of functional hepatocytes. This was further confirmed by high expression liver detoxification CYP genes and proteins and fatty acid oxidation in the subtype. However, adjacent cirrhotic tissues showed stronger HepPar1/Arg1 staining than tumor tissues even in the hepatocyte-like subtype, suggesting oncogenic progresses may have disrupted normal hepatocyte differentiation and function. Since liver damage from viral infections, excessive alcohol consumption or steatosis is almost invariably preceding oncogenic transformation of hepatocellular carcinoma, a potential mechanism underlying lineage association of cell lineage associated subtypes could be dysregulation and disruption of hepatocyte differentiation, de-differentiation or transdifferentiation programs during liver damage repair. YES-associated protein (YAP) activation, a molecular feature of cholangio-like hepatocellular carcinoma, was found to be able to subvert hepatocyte differentiation and gear them towards biliary cell lineage. Cells of origin can also be a contributor to cell lineage heterogeneity among subtypes, although a cell lineage tracing study showed that hepatocytes but not other epithelial cell types are the only source of hepatocellular carcinoma in mouse models. The third potential mechanism of lineage heterogeneity could also be driven by oncogenesis occurring in hepatocytes from different liver lobule metabolic zones. For example, the high immune presence in cholangio-like echos the periportal concentration of innate and adaptive immune cells mediated by liver sinusoidal endothelial cells and a CXCL9 cytokine gradient. While the causal mechanisms of lineage association of molecular subtypes remains elusive and needs further validation in high-resolution single cell genomic data, protein markers for known hepatic epithelial cell types, e.g. HepPar1, Arg1, CK19 or GPC3, may be used to design multiplex immunohistochemistry assays for subtype classification.

As noted, the three molecular subtypes exhibited a differential response to combination immunotherapy, such as atezolizumab (anti-PD-L1) plus bevacizumab (anti-VEGF) combination therapy. In particular, the cholangio-like subtype is characterized with high immune cell infiltration and pre-existing immunity, which might explain the favorable response of patients in this subtype to anti-PD-L1 plus anti-VEGF combination therapy. Indeed, pre-existing immunity has been shown to be associated with better response to combination immunotherapy in both GO30140 group A and IMbrave150 Atezo+Bev group (HCC primary biomarker paper). On the other hand, the progenitor-like subtype has high expression of oncofetal genes GPC3 and AFP and a desertic immune milieu that may confer resistance to Atezo+Bev. In fact, GPC3 is among the top 10 significantly upregulated genes in TCGA hepatocellular carcinoma as compared to normal tissue and associated with poorer prognosis, further highlighting its importance as a new therapeutic target. In addition, the S2 subclass, an overlapping subclass of hepatocellular carcinoma as the progenitor-like, was associated with the poorest prognosis, further highlighting that this subpopulation of patients really have a high unmet medical need and limited therapeutic options. To that end, the bi-specific antibody ERY974 against GPC3 and CD3 T-cells exhibits the ability to recruit and activate T cells from periphery to GPC3 expressing tumors in huNOG xenograft models and resulted in a strong tumor cell killing. Accordingly, the addition of an anti-GPC3/anti-CD3 bispecific antibody (ERY974) may overcome subtype specific resistance to combination immunotherapy associated with the progenitor-like subtype.

Through a survey of the mutation landscape, TP53 and CTNNB1 mutations are identified as being distinctively associated with subtypes. Hepatocyte-like enriches for CTNNB1 activating mutations while the other two have more TP53 mutations. WNT/β-catenin signaling activation have been previously linked to immune desert phenotype and resistance to immunotherapies in hepatocellular carcinoma and potential acquired resistance to ICI in metastatic melanoma. However, in the IMbrave 150 study, similar survival benefit from combination immunotherapy (e.g., atezolizumab (anti-PD-L1) plus bevacizumab (anti-VEGF) combination therapy) were observed in patients with wild type or mutant CTNNB1 indicating that adding anti-VEGF may overcome WNT/β-catenin signaling mediated resistance to ICI such as atezolizumab (HCC primary biomarker paper). This hypothesis was further validated in an ICI resistant hepatocellular carcinoma mouse model driven by β-catenin activation where anti-PD-L1 plus anti-VEGF overcame resistance to ICI. This may also explain why clinical activity of atezolizumab (anti-PD-L1) plus bevacizumab (anti-VEGF) combination therapy is still present in the hepatocyte-like subtype independent of CTNNB1 alterations and lack of immune presence.

FIG. 13 depicts a flowchart illustrating an example of a process 1400 for hepatocellular carcinoma (HCC) subtype classification and treatment, in accordance with some example embodiments. Referring to FIG. 1-14, the process 1400 may be performed at the digital pathology platform 110, for example, by the analysis engine 115.

At 1402, the analysis engine 115 may identify, for a liver epithelial cell lineage, one or more features associated with the liver epithelial cell lineage. As noted, hepatocellular carcinoma (HCC) is a highly heterogeneous disease with complex etiological factors as well as diverse molecular and cellular dysfunctions. Individual hepatocellular carcinoma molecular subtypes, such as the cholangio-like subtype, the progenitor-like subtype, and the hepatocyte-like subtype, may be identified and validated based on genetic features that evidence a strong linkage to different liver epithelia cell lineages. Moreover, the association between hepatocellular carcinoma molecular subtypes and liver epithelial cell lineage may be further characterized by distinct tumor-intrinsic features and tumor microenvironment (TME) features, with each hepatocellular carcinoma molecular subtype exhibiting a unique combination of tumor-intrinsic features and tumor microenvironment features.

In some example embodiments, individual hepatocellular carcinoma molecular subtypes may be identified and validated based on transcriptome data associated with different various hepatocellular carcinoma tumor samples. For example, the cholangio-like, progenitor-like, and hepatocyte-like subtypes may be identified by applying, to the transcriptome data, a cluster analysis (such as non-negative matrix factorization (NMF)), a classifier (such as a random forest classifier), and/or the like. In the case of cluster analysis, the analysis engine 115 may iteratively group the transcriptome data to identify the most robust clustering pattern, which includes an optimal quantity of clusters in which intra-cluster correlation between members of each cluster is maximized. The resulting clusters, which corresponds to the cholangio-like subtype, the progenitor-like subtype, and the hepatocyte-like subtype, may exhibit a maximum cophenetic correlation value (0.98).

At 1404, the analysis engine 115 may designate the one or more features as representative of a molecular subtype of hepatocellular carcinoma. In some example embodiments, each molecular subtype of hepatocellular carcinoma (HCC) may be associated with a distinct combination of tumor cell-intrinsic features and tumor microenvironment (TME) features. For example, in addition to the linkage to a particular liver epithelial cell lineage, each molecular subtype may exhibit different tumor cell-intrinsic features such as immunohistochemistry of cytochromes P450, expression level of cytochromes P450, Hippo signaling pathway, expression level of YES-associated protein (YAP), and/or the like. Alternatively and/or additionally, each molecular subtype may also exhibit different tumor microenvironment (TME) features including, for example, quantity of fibroblast activation protein in stroma, vessel density, density of cluster of differentiate 8 (CD8) in epitumor, quantity of MHCI+ tumor cells, density of cluster of differentiate 8 (CD8) in epitumor, density of PDL1+, density of activated T cells, density of exhausted T cells, and/or the like.

At 1406, the analysis engine 115 may receive a tumor sample of a patient. In some example embodiments, the analysis engine 115 may receive, from the imaging system 120 and/or the client device 130, a variety of data associated with a hepatocellular carcinoma (HCC) tumor sample of a patient. For example, in some instances, the analysis engine 115 may receive transcriptome data associated with the hepatocellular carcinoma tumor sample. Alternatively and/or additionally, the analysis engine 115 may receive one or more images (e.g., whole slide images) of the hepatocellular carcinoma tumor sample.

At 1408, the analysis engine 115 may determine, based at least on the one or more features being detected within the tumor sample of the patient, that the patient exhibits the molecular subtype associated with hepatocellular carcinoma. In some example embodiments, the hepatocellular carcinoma (HCC) molecular subtype exhibited by the patient may be determined based at least on one or more features present within the hepatocellular carcinoma tumor sample of the patient. For example, in cases where the analysis engine 115 receives transcriptome data associated with the hepatocellular carcinoma tumor sample, the analysis engine 115 may apply a cluster analysis or a classifier to determine, based at least on one or more genetic features present within the transcriptome data, the hepatocellular carcinoma (HCC) molecular subtype exhibited by the patient. Alternatively and/or additionally, the analysis engine 115 may receive one or more images of the hepatocellular carcinoma tumor sample (e.g., whole slide images), in which case the hepatocellular carcinoma molecular subtype of the patient may be identified by analyzing the one or more images to detect one or more of tumor cell-intrinsic features and/or tumor microenvironment features (TME).

At 1410, the analysis engine 115 may determine, based at least on the molecular subtype of the patient, a treatment for hepatocellular carcinoma. As noted, each molecular subtype of hepatocellular carcinoma may be associated with a unique immune landscape, which may give rise to subtype-specific responses to combination immunotherapies such as atezolizumab (anti-PD-L1) plus bevacizumab (anti-VEGF) combination therapy and/or the like. For example, the cholangio-like subtype, and to a lesser extent the hepatocyte-like subtype, may be more responsive to a atezolizumab (anti-PD-L1) plus bevacizumab (anti-VEGF) combination therapy and/or the like. Contrastingly, the progenitor-like subtype is associated with a subtype-specific resistance atezolizumab (anti-PD-L1) plus bevacizumab (anti-VEGF) combination therapy and/or the like.

Accordingly, treatment for the patient may be determined based at least on the molecular subtype present in the hepatocellular carcinoma (HCC) tumor sample of the patient. In the event the patient is identified as exhibiting a hepatocellular carcinoma molecular subtype without any subtype-specific resistance to combination immunotherapy (e.g., the cholangio-like subtype or the hepatocyte-like subtype), the analysis engine 115 may determine a treatment that includes a combination immunotherapy such as atezolizumab (anti-PD-L1) plus bevacizumab (anti-VEGF) combination therapy and/or the like. Alternatively, where the patent is identified as exhibiting a hepatocellular carcinoma molecular subtype having a subtype-specific resistance to combination immunotherapy, the analysis engine 115 may determine a treatment that includes, in addition to a combination immunotherapy (e.g., a atezolizumab (anti-PD-L1) plus bevacizumab (anti-VEGF) combination therapy), an additional therapy (e.g., a biopharmaceutical such as an GPC3/CD3 bi-specific antibody) to overcome the subtype-specific resistance to the combination immunotherapy.

FIG. 14 depicts a block diagram illustrating an example of computing system 1500, in accordance with some example embodiments. Referring to FIGS. 1 and 15, the computing system 1500 may be used to implement the digital pathology platform 110, the client device 130, and/or any components therein.

As shown in FIG. 14, the computing system 1500 can include a processor 1510, a memory 1520, a storage device 1530, and input/output devices 1540. The processor 1510, the memory 1520, the storage device 1530, and the input/output devices 1540 can be interconnected via a system bus 1550. The processor 1510 is capable of processing instructions for execution within the computing system 1500. Such executed instructions can implement one or more components of, for example, the digital pathology platform 110, the client device 130, and/or the like. In some example embodiments, the processor 1510 can be a single-threaded processor. Alternately, the processor 1510 can be a multi-threaded processor. The processor 1510 is capable of processing instructions stored in the memory 1520 and/or on the storage device 1530 to display graphical information for a user interface provided via the input/output device 1540.

The memory 1520 is a computer readable medium such as volatile or non-volatile that stores information within the computing system 1500. The memory 1520 can store data structures representing configuration object databases, for example. The storage device 1530 is capable of providing persistent storage for the computing system 1500. The storage device 1530 can be a floppy disk device, a hard disk device, an optical disk device, or a tape device, or other suitable persistent storage means. The input/output device 1540 provides input/output operations for the computing system 1500. In some example embodiments, the input/output device 1540 includes a keyboard and/or pointing device. In various implementations, the input/output device 1540 includes a display unit for displaying graphical user interfaces.

According to some example embodiments, the input/output device 1540 can provide input/output operations for a network device. For example, the input/output device 1540 can include Ethernet ports or other networking ports to communicate with one or more wired and/or wireless networks (e.g., a local area network (LAN), a wide area network (WAN), the Internet).

In some example embodiments, the computing system 1500 can be used to execute various interactive computer software applications that can be used for organization, analysis and/or storage of data in various formats. Alternatively, the computing system 1500 can be used to execute any type of software applications. These applications can be used to perform various functionalities, e.g., planning functionalities (e.g., generating, managing, editing of spreadsheet documents, word processing documents, and/or any other objects, etc.), computing functionalities, communications functionalities, etc. The applications can include various add-in functionalities or can be standalone computing products and/or functionalities. Upon activation within the applications, the functionalities can be used to generate the user interface provided via the input/output device 1540. The user interface can be generated and presented to a user by the computing system 1500 (e.g., on a computer screen monitor, etc.).

One or more aspects or features of the subject matter described herein can be realized in digital electronic circuitry, integrated circuitry, specially designed ASICs, field programmable gate arrays (FPGAs) computer hardware, firmware, software, and/or combinations thereof. These various aspects or features can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device. The programmable system or computing system may include clients and servers. A client and server are generally remote from each other and typically interact through a communication network. The relationship of client and server arises by virtue of computer programs running on the respective computers and having a client-server relationship to each other.

These computer programs, which can also be referred to as programs, software, software applications, applications, components, or code, include machine instructions for a programmable processor, and can be implemented in a high-level procedural and/or object-oriented programming language, and/or in assembly/machine language. As used herein, the term “machine-readable medium” refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. The machine-readable medium can store such machine instructions non-transitorily, such as for example as would a non-transient solid-state memory or a magnetic hard drive or any equivalent storage medium. The machine-readable medium can alternatively or additionally store such machine instructions in a transient manner, such as for example, as would a processor cache or other random access memory associated with one or more physical processor cores.

To provide for interaction with a user, one or more aspects or features of the subject matter described herein can be implemented on a computer having a display device, such as for example a cathode ray tube (CRT) or a liquid crystal display (LCD) or a light emitting diode (LED) monitor for displaying information to the user and a keyboard and a pointing device, such as for example a mouse or a trackball, by which the user may provide input to the computer. Other kinds of devices can be used to provide for interaction with a user as well. For example, feedback provided to the user can be any form of sensory feedback, such as for example visual feedback, auditory feedback, or tactile feedback; and input from the user may be received in any form, including acoustic, speech, or tactile input. Other possible input devices include touch screens or other touch-sensitive devices such as single or multi-point resistive or capacitive track pads, voice recognition hardware and software, optical scanners, optical pointers, digital image capture devices and associated interpretation software, and the like.

### Embodiments

Among the provided embodiments are:

1. A computer-implemented method, comprising:
identifying, for a liver epithelial cell lineage, one or more features associated with the liver epithelial cell lineage;
designating the one or more features as representative of a molecular subtype associated with hepatocellular carcinoma (HCC);
receiving a tumor sample of a patient; and
determining, based on the one or more features being detected within the tumor sample of the patient, that the patient exhibits the molecular subtype associated with hepatocellular carcinoma.

2. The computer-implemented method of Embodiment 1, wherein the one or more features comprise genetic features.

3. The computer-implemented method of Embodiment 2, wherein the operations further comprise:
identifying, based at least on transcriptome data associated with a plurality of hepatocellular carcinoma (HCC) tissue samples, a plurality of molecular subtypes associated with hepatocellular carcinoma (HCC).

4. The computer-implemented method of Embodiment 3, wherein the plurality of subtypes are identified by applying, to the transcriptome data, a cluster analysis to identify a quantity of subpopulations present within the transcriptome data.

5. The computer-implemented method of Embodiment 4, wherein the cluster analysis is applied to identify one or more subpopulations associated with a maximum cophenetic correlation value.

6. The computer-implemented method of any one of Embodiments 4 to 5, wherein the cluster analysis comprises a non-negative matrix factorization (NMF).

7. The computer-implemented method of any one of Embodiments 4 to 6, wherein the cluster analysis includes one or more of a connectivity-based clustering, a centroid-based clustering, a distribution-based clustering, a density-based clustering, a subspace-based clustering, a group-based clustering, and a graph-based clustering.

8. The computer-implemented method of any one of Embodiments 3 to 7, wherein the plurality of subtypes are identified and/or validated by applying, to the transcriptome data, a classifier.

9. The computer-implemented method of Embodiment 8, wherein the classifier comprises a random forest classifier.

10. The computer-implemented method of any one of Embodiments 1 to 9, wherein the one or more features include at least one of a tumor-cell intrinsic feature and a tumor microenvironment feature.

11. The computer-implemented method of any one of Embodiments 1 to 10, wherein the one or more features include an immunohistochemistry of cytochromes P450, an expression level of cytochromes P450, a Hippo signaling pathway, and/or an expression level of YES-associated protein (YAP).

12. The computer-implemented method of any one of Embodiments 1 to 11, wherein the one or more features include a quantity of fibroblast activation protein in stroma, a vessel density, a density of cluster of differentiate 8 (CD8) in epitumor, a quantity of MHCI+ tumor cells, a density of cluster of differentiate 8 (CD8) in epitumor, a density of PDL1+, a density of activated T cells, and/or a density of exhausted T cells.

13. The computer-implemented method of any one of Embodiments 1 to 12, wherein the molecular subtype associated with hepatocellular carcinoma comprises a cholangio-like subtype, and wherein the liver epithelial cell lineage comprises cholangiocytes.

14. The computer-implemented method of any one of Embodiments 1 to 13, wherein the molecular subtype associated with hepatocellular carcinoma comprises a hepatocyte-like subtype, and wherein the liver epithelial cell linage comprises hepatocytes.

15. The computer-implemented method of any one of Embodiments 1 to 14, wherein the molecular subtype associated with hepatocellular carcinoma comprises a progenitor-like subtype, and wherein the liver epithelial cell lineage comprises bi-potent progenitors.

16. The computer-implemented method of any one of Embodiments 1 to 15, wherein the operations further comprise:
determining, based at least on the molecular subtype of the patient, a treatment for hepatocellular carcinoma (HCC).

17. The computer-implemented method of Embodiment 16, wherein the treatment for hepatocellular carcinoma (HCC) includes a combination immunotherapy based at least on the patient having a cholangio-like subtype or a hepatocyte-like subtype.

18. The computer-implemented method of any one of Embodiments 16 to 17, wherein the treatment for hepatocellular carcinoma (HCC) includes an atezolizumab (anti-PD-L1) plus bevacizumab (anti-VEGF) combination therapy based at least on the patient having a cholangio-like subtype or a hepatocyte-like subtype.

19. The computer-implemented method of any one of Embodiments 16 to 18, wherein the treatment for hepatocellular carcinoma (HCC) includes, based at least on the patient having a progenitor-like subtype, one or more additional therapies to overcome a subtype-specific resistance to combination immunotherapy associated with the progenitor-like subtype.

20. The computer-implemented method of any one of Embodiments 16 to 19, wherein the treatment for hepatocellular carcinoma (HCC) includes, based at least on the patient having a progenitor-like subtype, an GPC3/CD3 bi-specific antibody in addition to a combination immunotherapy.

21. The computer-implemented method of any one of Embodiments 1 to 20, wherein the one or more features include a cancer epithelium tissue, a necrosis tissue, and/or a normal tissue present in an image of the tumor sample.

22. The computer-implemented method of any one of Embodiments 1 to 21, wherein the one or more features include a growth pattern present in an image of the tumor sample.

23. The computer-implemented method of any one of Embodiments 1 to 22, wherein the one or more features include one or more cancer epithelial cells, fibroblast cells, endothelial cells, and normal cells present in an image of the tumor sample.

24. The computer-implemented method of any one of Embodiments 1 to 23, wherein the one or more features include one or more hepatocellular carcinoma (HCC) hepatocyte-like cancer epithelial cells, hepatocellular carcinoma (HCC) cancer epithelial cells with Mallory Hyaline or globules, and hepatocellular carcinoma (HCC) heptoblast-like cancer epithelial cells.

25. The computer-implemented method of any one of Embodiments 1 to 24, further comprising:
determining, based at least on the molecular subtype of the patient, a response and/or a pathological response to a treatment for the patient.

26. A system, comprising:
at least one data processor; and
at least one memory storing instructions, which when executed by the at least one data processor, result in operations comprising the method of any of one of Embodiments 1 to 25.

27. A non-transitory computer readable medium storing instructions, which when executed by at least one data processor, result in operations comprising any one of Embodiments 1 to 25.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” Use of the term “based on,” above and in the claims is intended to mean, “based at least in part on,” such that an unrecited feature or element is also permissible.

The subject matter described herein can be embodied in systems, apparatus, methods, and/or articles depending on the desired configuration. The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. For example, the implementations described above can be directed to various combinations and subcombinations of the disclosed features and/or combinations and subcombinations of several further features disclosed above. In addition, the logic flows depicted in the accompanying figures and/or described herein do not necessarily require the particular order shown, or sequential order, to achieve desirable results. Other implementations may be within the scope of the following claims.

What is claimed is:

1. A computer-implemented method, comprising:
identifying, for a liver epithelial cell lineage, one or more features associated with the liver epithelial cell lineage;
designating the one or more features as representative of a molecular subtype associated with hepatocellular carcinoma (HCC);
receiving a tumor sample of a patient; and
determining, based on the one or more features being detected within the tumor sample of the patient, that the patient exhibits the molecular subtype associated with hepatocellular carcinoma.

2. The computer-implemented method of claim 1, wherein the one or more features comprise genetic features.

3. The computer-implemented method of claim 2, wherein the operations further comprise:
identifying, based at least on transcriptome data associated with a plurality of hepatocellular carcinoma (HCC) tissue samples, a plurality of molecular subtypes associated with hepatocellular carcinoma (HCC).

4. The computer-implemented method of claim 3, wherein the plurality of subtypes are identified by applying, to the transcriptome data, a cluster analysis to identify a quantity of subpopulations present within the transcriptome data.

5. The computer-implemented method of claim 4, wherein the cluster analysis is applied to identify one or more subpopulations associated with a maximum cophenetic correlation value.

6. The computer-implemented method of claim 4, wherein the cluster analysis comprises a non-negative matrix factorization (NMF).

7. The computer-implemented method of claim 4, wherein the cluster analysis includes one or more of a connectivity-based clustering, a centroid-based clustering, a distribution-based clustering, a density-based clustering, a subspace-based clustering, a group-based clustering, and a graph-based clustering.

8. The computer-implemented method of claim 3, wherein the plurality of subtypes are identified and/or validated by applying, to the transcriptome data, a classifier.

9. The computer-implemented method of claim 8, wherein the classifier comprises a random forest classifier.

10. The computer-implemented method of claim 1, wherein the one or more features include at least one of a tumor-cell intrinsic feature and a tumor microenvironment feature.

11. The computer-implemented method of claim 1, wherein the one or more features include an immunohistochemistry of cytochromes P450, an expression level of cytochromes P450, a Hippo signaling pathway, and/or an expression level of YES-associated protein (YAP).

12. The computer-implemented method of claim 1, wherein the one or more features include a quantity of fibroblast activation protein in stroma, a vessel density, a density of cluster of differentiate 8 (CD8) in epitumor, a quantity of MHCI+ tumor cells, a density of cluster of differentiate 8 (CD8) in epitumor, a density of PDL1+, a density of activated T cells, and/or a density of exhausted T cells.

13. The computer-implemented method of claim 1, wherein the molecular subtype associated with hepatocellular carcinoma comprises a cholangio-like subtype, and wherein the liver epithelial cell lineage comprises cholangiocytes.

14. The computer-implemented method of claim 1, wherein the molecular subtype associated with hepatocellular carcinoma comprises a hepatocyte-like subtype, and wherein the liver epithelial cell linage comprises hepatocytes.

15. The computer-implemented method of claim 1, wherein the molecular subtype associated with hepatocellular carcinoma comprises a progenitor-like subtype, and wherein the liver epithelial cell lineage comprises bi-potent progenitors.

16. The computer-implemented method of claim 1, wherein the operations further comprise:
determining, based at least on the molecular subtype of the patient, a treatment for hepatocellular carcinoma (HCC).

17. The computer-implemented method of claim 16, wherein the treatment for hepatocellular carcinoma (HCC) includes a combination immunotherapy based at least on the patient having a cholangio-like subtype or a hepatocyte-like subtype.

18. The computer-implemented method of claim 16, wherein the treatment for hepatocellular carcinoma (HCC) includes an atezolizumab (anti-PD-L1) plus bevacizumab (anti-VEGF) combination therapy based at least on the patient having a cholangio-like subtype or a hepatocyte-like subtype.

19. The computer-implemented method of claim 16, wherein the treatment for hepatocellular carcinoma (HCC) includes, based at least on the patient having a progenitor-like subtype, one or more additional therapies to overcome a subtype-specific resistance to combination immunotherapy associated with the progenitor-like subtype.

20. The computer-implemented method of claim 16, wherein the treatment for hepatocellular carcinoma (HCC) includes, based at least on the patient having a progenitor-like subtype, an GPC3/CD3 bi-specific antibody in addition to a combination immunotherapy.

21. The computer-implemented method of claim 1, wherein the one or more features include a cancer epithelium tissue, a necrosis tissue, and/or a normal tissue present in an image of the tumor sample.

22. The computer-implemented method of claim 1, wherein the one or more features include a growth pattern present in an image of the tumor sample.

23. The computer-implemented method of claim 1, wherein the one or more features include one or more cancer epithelial cells, fibroblast cells, endothelial cells, and normal cells present in an image of the tumor sample.

24. The computer-implemented method of claim 1, wherein the one or more features include one or more hepatocellular carcinoma (HCC) hepatocyte-like cancer epithelial cells, hepatocellular carcinoma (HCC) cancer epithelial cells with Mallory Hyaline or globules, and hepatocellular carcinoma (HCC) heptoblast-like cancer epithelial cells.

25. The computer-implemented method of claim 1, further comprising:
determining, based at least on the molecular subtype of the patient, a response and/or a pathological response to a treatment for the patient.

26. A system, comprising:
at least one data processor; and
at least one memory storing instructions, which when executed by the at least one data processor, result in operations comprising the method of claim 1.

27. A non-transitory computer readable medium storing instructions, which when executed by at least one data processor, result in operations comprising that of claim 1.
